SEQUENCE LISTING (20 sequences)

SEQ ID NO: 1 - DNA, 21 bp, synthetic construct
  source 1..21: mol_type = other DNA
aaagtgtcccagccaagaaca

SEQ ID NO: 2 - DNA, 26 bp, synthetic construct
  source 1..26: mol_type = other DNA
ctctgtaggtctcactgcttcttttg

SEQ ID NO: 3 - DNA, 23 bp, synthetic construct
  source 1..23: mol_type = other DNA
tgatacgtgacagtttcccacaa

SEQ ID NO: 4 - DNA, 17 bp, synthetic construct
  source 1..17: mol_type = other DNA
ttgggcatcgggtcctt

SEQ ID NO: 5 - DNA, 18 bp, synthetic construct
  source 1..18: mol_type = other DNA
ccagcgactcgccagagt

SEQ ID NO: 6 - DNA, 19 bp, synthetic construct
  source 1..19: mol_type = other DNA
ggcgaaagccctcaatttc

SEQ ID NO: 7 - DNA, 22 bp, synthetic construct
  source 1..22: mol_type = other DNA
gatgggacgtacactgggagaa

SEQ ID NO: 8 - DNA, 21 bp, synthetic construct
  source 1..21: mol_type = other DNA
tccaagcaatggaatctggaa

SEQ ID NO: 9 - DNA, 21 bp, synthetic construct
  source 1..21: mol_type = other DNA
gagaacagctgcacccacttc

SEQ ID NO: 10 - DNA, 24 bp, synthetic construct
  source 1..24: mol_type = other DNA
cagctgatccttcatttgaaagaa

SEQ ID NO: 11 - DNA, 21 bp, synthetic construct
  source 1..21: mol_type = other DNA
ctgggacctacacctgccata

SEQ ID NO: 12 - DNA, 23 bp, synthetic construct
  source 1..23: mol_type = other DNA
aggatttgggagtcactgtgatg

SEQ ID NO: 13 - DNA, 21 bp, synthetic construct
  source 1..21: mol_type = other DNA
aaacaacagcagctccaacca

SEQ ID NO: 14 - DNA, 20 bp, synthetic construct
  source 1..20: mol_type = other DNA
cgatgatctcccctgcatct

SEQ ID NO: 15 - DNA, 26 bp, synthetic construct
  source 1..26: mol_type = other DNA
tcaatagcaacaaaaagaaacgagat

SEQ ID NO: 16 - DNA, 21 bp, synthetic construct
  source 1..21: mol_type = other DNA
catgtattgctttgcgttgga

SEQ ID NO: 17 - DNA, 20 bp, synthetic construct
  source 1..20: mol_type = other DNA
tgctgaggatttggaaaggg

SEQ ID NO: 18 - DNA, 21 bp, synthetic construct
  source 1..21: mol_type = other DNA
acagagggctacaatgtgatg

SEQ ID NO: 19 - DNA, 21 bp, synthetic construct
  source 1..21: mol_type = other DNA
gagagttctgggattgtaccg

SEQ ID NO: 20 - DNA, 20 bp, synthetic construct
  source 1..20: mol_type = other DNA
atcctcatgattaccgcagc

(19) United States
(12) Patent Application Publication
(10) Pub. No.: US 2024/0167095 A1
(43) Pub. Date: May 23, 2024

(54) METHODS AND COMPOSITIONS FOR PREDICTING TOLERANCE IN TRANSPLANT PATIENTS

(71) Applicant: Veritas Therapeutics Inc., Thornhill (CA)

(72) Inventors: Gary Levy, Thornhill (CA); Andrzej Chruscinski, Toronto (CA); Stephen Juvet, Toronto (CA)

(21) Appl. No.: 18/511,249

(22) Filed: Nov. 16, 2023

Related U.S. Application Data
(60) Provisional application No. 63/426,145, filed on Nov. 17, 2022.

(30) Foreign Application Priority Data
Nov. 17, 2022 (CA) 3182289

(51) Int. Cl. C12Q 1/6883

(52) U.S. Cl. CPC C12Q 1/6883; C12Q 2600/106; C12Q 2600/158

(57) ABSTRACT

Provided herein is a method of predicting operational tolerance in a transplant patent and/or identifying a transplant patient as a candidate for reducing the dosage of immunosuppressant, comprising determining the ratio of the expression level of an anti-inflammatory gene to the expression level of a pro-inflammatory gene in PBMCs from the patient. The method can further comprise determining the ratio in a sample of the graft of the patient. Also provided is a kit that can be used to practice the methods disclosed herein.

### CROSS-REFERENCE TO RELATED APPLICATIONS

This application claims the benefit of priority to U.S. Provisional Application No. 63/426,145 filed Nov. 17, 2022 and Canadian Patent Application No. 3,182,289 filed Nov. 17, 2022, the contents of both of which are incorporated herein by reference in their entirety.

### INCORPORATION OF SEQUENCE LISTING

A computer readable form of the Sequence Listing “25306-P69035US01_SequenceListing.xml” (18,668 bytes), submitted via Patent Center and created on Nov. 15, 2023, is herein incorporated by reference.

### FIELD

The present disclosure relates to methods for predicting operational tolerance in a transplant patient and/or identifying a transplant patient as a candidate for reducing the dosage of immunosuppressant.

### BACKGROUND

The outcomes of solid organ transplantation have improved over the past three decades, mainly as a result of advances in surgical techniques, management of post-transplant complications, and introduction of newer immunosuppressive agents [1]. However, the need for long-term immunosuppression (IS) is associated with serious transplant-related complications reducing long-term survival [2]. Strategies to reduce or stop IS remain an important goal to prevent long-term side effects.

It is known that rejection involves both elements of the innate and adaptive immunity and are similar in all transplanted organs. It is also known that immunosuppression can be stopped in solid organ transplant patients, however, discontinuation of immunosuppression can be done more frequently in patients who have undergone liver transplantation without the development of graft rejection—a phenomenon known as spontaneous operational tolerance [2,3]. It is known that kidney, heart and lung transplant patients are also able to develop tolerance but less frequently than liver transplanted patients [2,3]. For example, a recent report by Cheruki et al has shown that renal transplant patients who have a high ratio of IL 10 to IFNγ have superior graft function and longevity [32]. As patients who have their immunosuppression discontinued are no longer susceptible to IS-related toxicity, investigators have sought to determine the frequency and clinical predictors of operational tolerance especially in the setting of liver transplantation. Over the past 25 years, a number of clinical trials have focused on operational tolerance in adult liver transplantation [4-12]. In these studies, the overall frequency of operational tolerance was shown to vary from 5.6 to 62.5% with the best results in small trials using highly selected patients. The combined success of IS withdrawal in these studies was 30.8% (140/455) in agreement with a review on operational tolerance which concluded that 20-40% of liver transplant recipients may be operationally tolerant [13]. Clinical predictors of operational tolerance included greater time post-liver transplantation, older age, and male sex [10]. Greater time post-liver transplantation was also determined to be a predictor of tolerance in paediatric liver transplantation [14].

A variety of different cellular and transcriptional markers in the peripheral blood have been identified that discriminate between tolerant and non-tolerant transplant recipients, although some studies had a retrospective cross-sectional design, in which tolerant recipients weaned off IS were compared with immunosuppressed controls, leading to a potential bias in the analysis of immunological parameters [3, 15, 16]. A more recent study suggested that the gene expression profiling of the liver biopsy may be more accurate than blood-related biomarkers in predicting the outcome of IS withdrawal [17]. This gene biomarker, which includes genes involved in the regulation of iron homeostasis, has been studied in a multi-centre trial in Europe (LIFT trial: NCT02498977). Although there is no final report on the LIFT study, preliminary data does not support that use of LIFT will be useful in identifying patients who are tolerant. There remains a need for reliable biomarkers to predict the outcome of IS withdrawal. Another approach using molecular medicine which examines both levels of circulating donor DNA and intragraft gene expression in renal transplant patients This has not again proven to identify tolerant patients. Finally the use of ALLOMAP in heart transplant patients has only been used to rule out the presence of rejection.

### SUMMARY

In one aspect, the present disclosure provides a method of predicting operational tolerance in a transplant patient who is on immunosuppressant, the method comprising: determining a peripheral blood mononuclear cell (PBMC) ratio of the expression levels of an anti-inflammatory gene to a pro-inflammatory gene in PBMCs from the patient; wherein the anti-inflammatory gene is selected from the group consisting of FGL2, FOXP3, IL10, TIGIT, LAG3 and TGFB; wherein the pro-inflammatory gene is selected from the group consisting of IFNγ and GZMB; and wherein a PBMC ratio of ≥1 is indicative that the patient will achieve operational tolerance.

In another aspect, the present disclosure provides a method of identifying a transplant patient on an immunosuppressant as a candidate for reducing the dosage of the immunosuppressant, the method comprising: determining a peripheral blood mononuclear cell (PBMC) ratio of the expression levels of an anti-inflammatory gene to a pro-inflammatory gene in PBMCs from the patient; wherein the anti-inflammatory gene is selected from the group consisting of FGL2, FOXP3, IL10, TIGIT, LAG3 and TGFB; wherein the pro-inflammatory gene is selected from the group consisting of IFNγ and GZMB; wherein if the PBMC ratio is ≥1, then the patient is a candidate for reducing the dosage of the immunosuppressant.

In one embodiment, the transplant is a solid organ transplant, optionally a heart, kidney, pancreas, lung or liver transplant.

In one embodiment, the transplant is a liver transplant. In one embodiment, the transplant patient is previously diagnosed with hepatitis C virus (HCV) cirrhosis, alcoholic cirrhosis, autoimmune disease, genetic liver disease, fulminant hepatic failure (FHF), and/or non-alcoholic steatohepatitis (NASH).

In one embodiment, the method further comprises obtaining a blood sample from the patient prior to determining the PBMC ratio.

In one embodiment, the PBMCs are Tregs or transitional B cells.

In one embodiment, the PBMCs are purified by binding to affinity ligands and/or antibody coated nanoparticles prior to determining the PBMC ratio.

In one embodiment, determining the PBMC ratio comprises measuring the expression levels of the anti-inflammatory gene and the pro-inflammatory gene in PBMCs. In one embodiment, measuring the expression levels comprises performing quantitative PCR (qPCR), optionally ultrafast qPCR.

In one embodiment, the anti-inflammatory gene is FGL2. In one embodiment, the pro-inflammatory gene is IFNγ. In one embodiment, the PBMC ratio is a ratio of the expression levels of FGL2 to IFNγ in PBMCs.

In one embodiment, the method further comprises determining an intragraft ratio of an anti-inflammatory gene to a pro-inflammatory gene in a graft sample of the patient; wherein a PBMC ratio of ≥1 combined with an intragraft ratio, for example an intragraft ratio of FOXP3/IFNγ 1, is indicative that the patient will achieve operational tolerance.

In one embodiment, the method further comprises obtaining the graft sample from the patient. In one embodiment, the graft sample is a liver biopsy sample.

In one embodiment, the anti-inflammatory gene for the intragraft ratio is FOXP3. In one embodiment, the pro-inflammatory gene for the intragraft ratio is IFNγ. In one embodiment, the intragraft ratio is a ratio of the expression levels of FOXP3 to IFNγ in the graft sample.

In one embodiment, the PBMC ratio is a ratio of the expression levels of FGL2 to IFNγ in PBMCs, and wherein the intragraft ratio is a ratio of the expression levels of FOXP3 to IFNγ in the graft sample.

In one embodiment, the method further comprises reducing the dosage of the immunosuppressant in the patient. In one embodiment, reducing the dosage of immunosuppressant is complete cessation of immunosuppressant.

In yet another aspect, the present disclosure provides a kit comprising reagents for measuring the expression level of at least one anti-inflammatory gene, wherein the anti-inflammatory gene is selected from the group consisting of FGL2, FOXP3, IL10, TIGIT, LAG3 and TGFB; and reagents for measuring the expression level of at least one pro-inflammatory gene, wherein the pro-inflammatory gene is selected from the group consisting of IFNγ and GZMB.

In one embodiment, the kit further comprises reagents for measuring the expression level of one or more housekeeping genes.

In one embodiment, the reagents for measuring the expression levels of the genes are multiplex PCR reagents.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the present disclosure will now be described in relation to the drawings in which:

FIG. 1 is a diagram illustrating the trial design. In Phase 1, eligible patients had blood collected for PBMC gene expression profiling. In Phase 2, patients with a positive tolerance biomarker underwent IS withdrawal, following a baseline liver biopsy. Liver biopsies were performed, and blood was collected at various months post-IS withdrawal as indicated. M, months.

FIG. 2 is an illustration of a method of nucleic acid isolation, amplification, detection and analysis.

FIG. 3 is an illustration of a method of multiplex PCR.

FIG. 4 is a series of graphs showing the comparison of GeXP RT-PCR with qPCR for the eight genes in the GeXP assay. An R2 value is shown for each of the correlations.

FIGS. 5A-5B are heatmaps and graphs showing the correlation of different gene ratios with FGL2/I IFNγ in the peripheral blood. (A) Heatmaps showing change in patient order using FOXP3/IFNγ and TGFB1/IFNγ gene ratios to sort patients compared with FGL2/IFNγ. (B) Graph of the correlation between the different ratios and FGL2/IFNγ. Heatmaps and graphs include data from patients (n=69) at the time of enrollment.

FIGS. 6A-6D are images showing baseline liver histopathology. (A and B) Biopsies show preserved microscopic architecture with no evidence of liver injury/disease. (C) Biopsy shows mild portal and lobular mononuclear infiltrate (arrow) in keeping with mild immune mediated injury/idiopathic hepatitis. (D) Biopsy shows non-necrotizing granulomatous inflammation in portal tracts and lobules (arrowhead). Special stains for fungal elements and acid fast bacilli were negative. Liver biopsies were stained with hematoxylin and eosin (H&E). Scale bars: 200 μm.

FIG. 7 is a flowchart of patient enrolment.

FIG. 8 is a diagram showing the probability of patients being free of rejection from the initiation of IS withdrawal. Tick marks indicate patients who are operationally tolerant.

FIGS. 9A-9B are heatmaps and images showing liver biopsy histopathology in tolerant patients. (A) Evaluation of liver biopsies for arteriopathy, duct injury, fibrosis, interface hepatitis, and portal inflammation. The absolute score for each of these parameters is represented in the heatmap as shown in the scale. Arteriopathy, duct loss, and portal inflammation were scored from 0 to 3, whereas fibrosis was scored from 0 to 4. Scoring was performed using Banff criteria [21, 22]. All eight tolerant patients had biopsies at baseline (B) and end of trial at 12 months (12M) post-IS withdrawal. Some of the tolerant patients also had biopsies available for evaluation at 24 months (24M) and 36 months (36M) post-IS withdrawal. White spaces in the heatmap represent biopsies that were not available for evaluation. (B) Liver biopsies in two patients (Patient Tol1 and Patient Tol2) pre-IS withdrawal, 12 months post-IS withdrawal, and 24 months post-IS withdrawal. Patient Tol1 was noted to have mild macrovesicular steatosis at baseline (arrow). On post-IS withdrawal biopsies, there was persistent macrovesicular steatosis and no evidence of cellular infiltrates. Patient Tol2 developed a focal portal mononuclear cell infiltrate at 1 year post-IS withdrawal (arrowhead). At 2 years post-IS withdrawal, the infiltrate had largely resolved. Liver biopsies were stained with haematoxylin and eosin (H&E). Scale bars: 300 μm.

FIG. 10 is a series of heatmaps representing evaluation of liver biopsies from non-tolerant patients. The absolute score for arteriopathy, duct injury, fibrosis, interface hepatitis and portal inflammation is represented in the heatmap as shown in the scale. Arteriopathy, duct injury, interface inflammation and portal inflammation were scored from 0 to 3, whereas fibrosis was scored from 0 to 4. All six non-tolerant patients had biopsies at baseline (B) and at the time of rejection (Rej). Patients Non-Tol5 and Non-Tol6 received steroids prior to the liver biopsy.

FIG. 11 is a series of graphs showing the comparison of baseline PBMC FGL2/IFNγ gene ratio in tolerant and non-tolerant patients. Comparison is made either using patients who failed IS withdrawal (n=6) or patients who either failed IS withdrawal or had abnormal baseline liver biopsies (n=15). Symbols represent median, and bars represent IQR. The comparison was made using a Mann-Whitney U test.

FIGS. 12A-12B are graphs and images showing PBMC gene expression and Treg in the peripheral blood. (A) Change in PBMC target gene expression at 3, 6, 9, and 12 months post-IS withdrawal in operationally tolerant recipients. For each recipient, gene expression was normalized to pre-withdrawal gene expression. Symbols represent median, and bars represent IQR. Significance was determined with the Kruskal-Wallis test followed by Dunn's multiple comparisons post hoc test. (B) Quantification of Tregs with mass cytometry. Tregs in peripheral blood were identified based on expression of CD3+CD4+CD25+CD127low markers and are shown as a percentage of CD4+ T cells. PBMC were studied pre-IS withdrawal and 3-6 months post-IS withdrawal in operationally tolerant recipients (n=6). Lines represent individual patients. Significance was determined used a paired t-test. *P<0.05; **P<0.01. IQR, interquartile range; M, months.

FIGS. 13A-13C are graphs and diagrams showing baseline and longitudinal liver allograft gene expression. (A) Baseline FOXP3/IFNγ liver gene ratio in operationally tolerant patients (n=8) versus unsuccessful (n=6) IS withdrawal. Symbols represent median, and bars represent IQR. Significance was determined by a Mann-Whitney U test. (B) Baseline FOXP3/IFNγ liver gene ratio separated by a FOXP3/IFNγ<1 or FOXP3/IFNγ 1. (C) Change in liver target gene expression at 6 and 12 months post-IS withdrawal in operationally tolerant recipients. For each recipient, gene expression was normalized to pre-withdrawal gene expression. Symbols represent median, and bars represent IQR. Significance was determined with the Kruskal-Wallis test followed by Dunn's multiple comparisons post hoc test. *P<0.05; **P<0.01. IQR, interquartile range; M, months.

FIGS. 14A-14G are images and graphs showing immunophenotyping of the T-cell compartment in liver allograft biopsies at pre-withdrawal and 12 and 24 months post-IS withdrawal. (A) Left panel: representative histology from a tolerant patient after successful IS withdrawal. Co-staining for CD4, CD8, and FOXP3 of intrahepatic T-cell infiltration was performed in liver biopsy sections. Liver sinusoidal epithelial cells weakly express CD4 and can be distinguished from T cells by strength of CD4 expression, shape, and localization of cells. The white line surrounds areas of portal infiltrations and excludes lumen of veins, arteries, and bile ducts. Right panel: higher magnification of portal infiltrates with clear nuclear localization of the FOXP3 in CD4+ Tregs (white arrows). (B) Size of portal infiltrates in liver biopsies before after complete and successful IS weaning. (C) Portal infiltration density of CD4+ T cells. (D) Portal infiltration density of CD8+ T cells. (E) Portal infiltration density of CD4+FOXP3+ Tregs. (F) Portal Tregs/(CD4++CD8+) ratio. Symbols represent median, and bars represent IQR for patients at 12M (n=6) and patients at 24M (n=4). Significance was determined with the Kruskal-Wallis test followed by Dunn's multiple comparisons post hoc test. *P<0.05. (G) Mean relative changes of histological parameters during operational tolerance compared to pre-withdrawal biopsies. IQR, interquartile range; M, months.

FIGS. 15A-15B are diagrams showing the results of RNA-seq on pre-withdrawal liver biopsies. (A) Volcano plot of genes upregulated and downregulated in tolerant patients (n=8) versus non-tolerant patients with unsuccessful withdrawal/abnormal biopsy (n=6 unsuccessful, n=3 abnormal). (B) Heatmap display of the 16 genes with significant (Padj <0.05) differences. Rows represent genes, and columns represent different patient samples. Hierarchical clustering was used to sort both rows and columns. A relative colour scheme was used to convert the minimum and maximum values in each row to colours.

FIG. 16 shows a schematic of a MicroGEM Immunometer.

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

The terms “operational tolerance”, “operationally tolerant and the like as used herein refer to stable and acceptable transplant or graft function without immunosuppression. When a recipient of a transplant or graft achieves operational tolerance, the transplant or graft does not induce a significant immune response by the immune system of the recipient. In one embodiment, these terms refer a state where a recipient who has been off immunosuppression for a minimum of 1 year, had not experienced rejection in the past 1 year, and is currently without any clinical or histologic signs of rejection.

The terms “non-tolerance”, “non-tolerant”, “not tolerated” and the like as used herein refer to the state where a transplant recipient is reliant on immunosuppression to avoid rejection. The terms also refer to a state where the transplant or graft induces an immune response by the immune system of the recipient.

The term “transplant” as used herein refers to the transplantation of a part of an organism, such as an organ, obtained from one source (the donor) to a recipient and also refers to the part of the organism transplanted. “Graft” is another term used to refer to the part of the organism transplanted. The donor may be a deceased donor or a living donor. The source of the transplant may be artificial, may be obtained from the same species as the recipient (allotransplant) or may be from a different species from the recipient (xenotransplant). Examples of transplants include solid organ transplants including, but not limited to, liver transplants, heart transplants, kidney transplants and lung transplants.

The term “immunosuppressant” as used herein refers to a medication or treatment a transplant recipient is receiving to suppress their immune response to the transplant. Examples of immunosuppressive therapies include, but are not limited to, cyclosporine, tacrolimus, mycophenolate mofetil, azathioprine (Imuran), anti-thymocyte globulin (ATG), OKT3 (muromonab-CD3), OKT4, sirolimus (rapamycin), everolimus and prednisone.

As used herein, the term “anti-inflammatory” refers to having an inhibitory effect on the inflammatory response of the immune system. The term is intended to be broad and encompasses any mechanism that inhibits, reduces, counteract and/or abolishes the inflammatory response.

As used herein, the term “pro-inflammatory” refers to having an enhancing effect on the inflammatory response of the immune system. The term is intended to be broad and encompasses any mechanism that increases, promotes, drives and/or amplifies the inflammatory response. As used herein, the term peripheral blood mononuclear cells (PBMCs) refers to peripheral blood cells having a round nucleus. PBMCs include undifferentiated PBMCs, lymphocytes (T cells, B cells, NK cells) and monocytes. The term PBMCs includes subsets of PBMCs including, for example, regulatory T cells (Tregs) and transitional B cells (TrB cells).

As used herein, the term “FGL2” refers to fibrinogen like 2 or fibroleukin, including FGL2 from any species or source and including isoforms, analogs, variants or functional derivatives of such a FGL2 gene or protein. The term also includes sequences that have been modified from any of the known published sequences of FGL2 genes or proteins. The FGL2 gene or protein may have any of the known published sequences for FGL2 which can be obtained from public sources such as GenBank. Examples of such sequences include, but are not limited to, Accession Nos. NM_006682.2.

As used herein, the term “FOXP3” refers to forkhead box P3, including FOXP3 from any species or source and including isoforms, analogs, variants or functional derivatives of such a FOXP3 gene or protein. The term also includes sequences that have been modified from any of the known published sequences of FOXP3 genes or proteins. The FOXP3 gene or protein may have any of the known published sequences for FOXP3 which can be obtained from public sources such as GenBank. Examples of such sequences include, but are not limited to, Accession Nos. NM_014009.3, NM_001114377.1, XM_017029566.1, XM_017029565.1, XM_006724533.2.

As used herein, the term “TIGIT” refers to T-cell immunoreceptor with Ig and ITIM domains, including TIGIT from any species or source and including isoforms, analogs, variants or functional derivatives of such a TIGIT gene or protein. The term also includes sequences that have been modified from any of the known published sequences of TIGIT genes or proteins. The TIGIT gene or protein may have any of the known published sequences for TIGIT which can be obtained from public sources such as GenBank. An example of such a sequence includes, but is not limited to, Accession No. NM_173799.3.

As used herein, the term “TGFB” or “TGFB1” refers to transforming growth factor beta-1 proprotein, including TGFB from any species or source and including isoforms, analogs, variants or functional derivatives of such a TGFB gene or protein. The term also includes sequences that have been modified from any of the known published sequences of TGFB genes or proteins. The TGFB gene or protein may have any of the known published sequences for TGFB which can be obtained from public sources such as GenBank. An example of such a sequence includes, but is not limited to, Accession Nos. NM_000660.6, XM_011527242.1.

As used herein, the term “IL10” refers to interleukin 10, including IL10 from any species or source and including isoforms, analogs, variants or functional derivatives of such a IL10 gene or protein. The term also includes sequences that have been modified from any of the known published sequences of IL10 genes or proteins. The IL10 gene or protein may have any of the known published sequences for IL10 which can be obtained from public sources such as GenBank. An example of such a sequence includes, but is not limited to, Accession Nos. NM_000572.2, XM_011509506.1.

As used herein, the term “LAG3” refers to lymphocyte activating 3, including LAG3 from any species or source and including isoforms, analogs, variants or functional derivatives of such a LAG3 gene or protein. The term also includes sequences that have been modified from any of the known published sequences of LAG3 genes or proteins. The LAG3 gene or protein may have any of the known published sequences for LAG3 which can be obtained from public sources such as GenBank. An example of such a sequence includes, but is not limited to, Accession Nos. NM_002286.5, XM_011520956.1.

As used herein, the term “IFNγ” or “IFN-γ” refers to interferon gamma, including IFNγ from any species or source and including isoforms, analogs, variants or functional derivatives of such an IFNγ gene or protein. The term also includes sequences that have been modified from any of the known published sequences of IFNγ genes or proteins. The IFNγ gene or protein may have any of the known published sequences for IFNγ which can be obtained from public sources such as GenBank. An example of such a sequence includes, but is not limited to, Accession No. NM_000619.2.

As used herein, the term “GZMB” refers to granzyme B, including GZMB from any species or source and including isoforms, analogs, variants or functional derivatives of such a GZMB gene or protein. The term also includes sequences that have been modified from any of the known published sequences of GZMB genes or proteins. The GZMB gene or protein may have any of the known published sequences for GZMB which can be obtained from public sources such as GenBank. An example of such a sequence includes, but is not limited to, Accession Nos. NM_004131.5, NM_001346011.1.

The terms “level”, “expression level”, “level of expression” and the like as used herein refers to the measurable quantity of a gene product produced by the gene in a sample of a patient, wherein the gene product can be a transcriptional product or a translated transcriptional product. Accordingly, the expression level can pertain to a nucleic acid gene product such as RNA or cDNA or a polypeptide gene product. The expression level can for example be detected de novo or correspond to a previous determination. The expression level can be determined or measured for example, using microarray methods, PCR methods, and/or antibody based methods, as is known to a person of skill in the art.

The term “PBMC ratio” as used herein refers to the ratio of the expression level of a gene to the expression level of another gene measured in peripheral blood mononuclear cells (PBMCs).

The term “intragraft ratio” as used herein refers to the ratio of the expression level of a gene to the expression level of another gene measured in a sample of the graft obtained from a transplant patient.

As used herein, the terms “reducing the dosage of immunosuppressant” and the like refer to reducing the amount of the immunosuppressant administered to a transplant patient and can encompass reducing the dosage and/or frequency of administration. The terms also encompass complete cessation of the use of the immunosuppressant in the patient.

The term “subject”, also referred as patient, as used herein includes all members of the animal kingdom including mammals, and suitably refers to humans. In one embodiment, a subject is a patient who has undergone a transplant.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 20%, 5-20%, or 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The definitions and embodiments described in particular sections are intended to be applicable to other embodiments herein described for which they are suitable as would be understood by a person skilled in the art.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

Further, the definitions and embodiments described in particular sections are intended to be applicable to other embodiments herein described for which they are suitable as would be under-stood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Although any methods and materials similar or equivalent to those described herein can also be used in the practice or testing of the present disclosure, examples of methods and materials are now described.

### II. Methods

It is demonstrated herein that the ratio of the expression levels of certain anti-inflammatory genes to certain pro-inflammatory genes in peripheral blood mononuclear cells (PBMCs) is different in liver transplant patients who achieved operational tolerance from those who developed rejection after immunosuppression withdrawal.

Accordingly, an aspect of the present disclosure provides a method of predicting operational tolerance in a transplant patient, the method comprising determining the ratio of the expression level of an anti-inflammatory gene to the expression level of a pro-inflammatory gene in PBMCs from the patient, wherein an anti-inflammatory to pro-inflammatory ratio of ≥1 is indicative of operational tolerance.

It is further demonstrated herein that a ratio measured from PBMCs, when combined with a ratio measured from an intragraft sample, further improves accuracy in predicting operational tolerance.

Accordingly, in some embodiments, the method further comprises measuring the expression levels of the anti-inflammatory and pro-inflammatory genes in an intragraft sample of the patient.

In an embodiment, where the PBMC ratio is ≥1, the patient is predicted to be able to achieve operational tolerance. In another embodiment, where the PBMC ratio is ≥1 and the intragraft ratio is ≥1, the patient is predicted to be able to achieve operational tolerance.

Where a patient is predicted to be able to achieve operational tolerance, the method may further comprise reducing the dosage of the immunosuppressant in the patient.

In another aspect, the present disclosure provides a method to identify a transplant patient on an immunosuppressant as a candidate for reducing the dosage of the immunosuppressant, the method comprising determining the ratio of the expression level of an anti-inflammatory gene to the expression level of a pro-inflammatory gene in PBMCs from the patient, wherein if the PBMC ratio is ≥1, then the patient is a candidate for reducing the dosage of the immunosuppressant.

In one embodiment, the method further comprises measuring the expression levels of the anti-inflammatory and pro-inflammatory genes in an intragraft sample of the patient.

In an embodiment, where the PBMC ratio is ≥1, then the patient is a candidate for reducing the dosage of the immunosuppressant. In another embodiment, where the PBMC ratio is ≥1 and the intragraft ratio is ≥1, then the patient is a candidate for reducing the dosage of the immunosuppressant. In a further embodiment, where the PBMC ratio is ≥1 and the intragraft ratio is ≥1, then the patient has achieved a state of tolerance and immunosuppression can be discontinued.

In another embodiment, where the PBMC ratio is ≥1 and intragraft Tregs are increased and the ratio of FOXP3/IFNγ is ≥1, then the patient has achieved a state of tolerance and immunosuppression can be discontinued.

Where the patient is identified to be a candidate for reducing the dosage of the immunosuppressant, the method may further comprise reducing the dosage of the immunosuppressant in the patient.

In one embodiment, the transplant is a liver transplant, a lung transplant, a kidney transplant, or a heart transplant.

In one embodiment, the transplant patient is a liver transplant patient.

The liver transplant patient may have been diagnosed with any liver disease or condition that led to the liver transplant, such as hepatitis C virus (HCV) cirrhosis, alcoholic cirrhosis, autoimmune disease, genetic liver disease, fulminant hepatic failure (FHF), primary sclerosing cholangitis, primary biliary cirrhosis, hepatocellular carcinoma hepatitis B virus infection (HBV) and non-alcoholic steatohepatitis (NASH).

In one embodiment, the anti-inflammatory gene is selected from the group consisting of FGL2, FOXP3, IL10, TIGIT, LAG3 and TGFB. In another embodiment, the anti-inflammatory gene is FGL2. In another embodiment, the anti-inflammatory gene is FOXP3. In one embodiment, the anti-inflammatory gene is IL10. In another embodiment, the anti-inflammatory gene is TIGIT. In another embodiment, the anti-inflammatory gene is LAG3. In one embodiment, the anti-inflammatory gene is TGFB.

In one embodiment, the pro-inflammatory gene is selected from the group consisting of IFNγ and GZMB. In another embodiment, the pro-inflammatory gene is IFNγ. In another embodiment, the pro-inflammatory gene is GZMB.

In one embodiment, the anti-inflammatory gene is FGL2 and the pro-inflammatory gene is IFNγ. In another embodiment, the PBMC ratio is a ratio of the expression level of FGL2 to the expression level of IFNγ.

In one embodiment, the anti-inflammatory gene is FOXP3 and the pro-inflammatory gene is IFNγ. In another embodiment, the intragraft ratio is a ratio of the expression level of FOXP3 to the expression level of IFNγ.

In one embodiment, the PBMC ratio is a ratio of the expression level of FGL2 to the expression level of IFNγ and the intragraft ratio is a ratio of the expression level of FOXP3 to the expression level of IFNγ.

The method disclosed herein may be used with patients who are receiving any type of immunosuppressant. Examples of immunosuppressants include, but are not limited to, tacrolimus, cyclosporine, mycophenolate mofetil (MMF), sirolimus (rapamycin), everolimus and prednisone.

Reducing the dosage of immunosuppressant may involve lowering the amount of the immunosuppressant administered and/or the frequency of administration. Typically, the dosage is reduced gradually over a period of time until complete cessation of the use of immunosuppressant and the patient is monitored for signs of graft rejection. For example, cyclosporin A (CsA) may be administered at a starting dose of 200 mg b.i.d. Dosage may then be reduced stepwise as follows: 200 mg q.a.m.; 100 mg q.h.s.; 100 mg b.i.d.; 50 mg b.i.d.; 50 mg q.d.; then complete cessation. The method disclosed herein may be used with any protocol for withdrawing or reducing immunosuppressant. For example, the dosage may be reduced over a period of 3 to 4 months in a stepwise fashion. Dosage may be reduced by, for example, 25% each month, so immunosuppressant can be completely withdrawn in 4 months. If complete withdrawal of immunosuppressant is not achievable, some level of reduction may also be beneficial and can still be a desired outcome.

In one embodiment, the method further comprises obtaining a blood sample from the patient for isolation of PBMCs. PBMCs can be isolated from peripheral blood using any suitable method known in the art, such as density gradient centrifugation. In density gradient centrifugation, mononuclear cells can be separated from other cell types based on differences in density. Suitable density gradient media include for example Ficoll™. PBMCs may also be isolated by depletion of other cell types from a blood sample. Commercial products such as MACSprep™ (Miltenyi Biotec) and EasySep™ (STEMCELL Technologies) can be used.

In another embodiment, PBMCs are isolated (or captured) using nanoparticles. In a further embodiment, Tregs (CD4⁺ CD25⁺) and Transitional B cells (CD19⁺CD24hiCD38hi) can be isolated from undifferentiated PBMCs using antibodies or affinity ligands tethered to nanoparticles.

In one embodiment, the method comprises first obtaining an intragraft sample. In an embodiment, the intragraft sample is a biopsy sample. A biopsy sample may be obtained, for example, percutaneously. In an embodiment, the intragraft sample is a liver biopsy sample. The biopsy optionally has a minimum core size of 2.5 cm and may be a percutaneous biopsy.

Expression of target genes can be measured by any suitable method. For example, transcript levels can be measured.

Transcript levels can be measured for example by quantitative PCR and/or hybridization-based methods (e.g. microarray). These methods are well known in the art. Expression of any suitable variant and/or mutant form of a target gene may be used. Typically, the measurement from a gene is normalized to one or more reference genes (e.g. housekeeping genes) to obtain the expression level. Examples of housekeeping genes that have been known to express at consistent mRNA level in PBMC and tissues include, for example, hypoxanthine-guanine phosphoribosyltransferase (HPRT), TATA box binding protein (TBP), beta-2 microglobulin (B2M), cancer susceptibility candidate-3 (CASC3), and ezrin (EZR). Expression levels of target genes may also be obtained from a database comprising expression data of a larger collection of genes, for example, from sequencing of the transcriptome or exome.

In one embodiment, the expression levels of the target genes are measured by quantitative PCR. Methods to design, test and optimize quantitative PCR to measure the expression levels of target genes are well known in the art. Multiplex PCR can be used to amplify multiple products in a single reaction. Commercial multiplex PCR assays can be used with the methods disclosed herein, for example, GenomeLab Gene Expression Profiler (GeXP) multiplex PCR assay.

In another embodiment, the expression levels of the target genes are measured by ultrafast quantitative PCR. Commercial qPCR assays can be used with the methods disclosed herein, for example, MicroGem's SAL6830 cartridge and system.

### III. Kits

The present disclosure also provides kits for practicing the methods disclosed herein.

In an embodiment, the kit comprises reagents for measuring the expression levels of at least one of the anti-inflammatory genes and at least one of the pro-inflammatory genes disclosed herein. The kit can further comprise reagents for measuring the expression levels of one of more housekeeping genes. The reagents can comprise primers specific for the genes and other components for performing quantitative PCR, such as buffer and enzymes. The reagents can also comprise components for reverse transcription of RNA. One or more components may be supplied in the form of a master mix. The kit may further comprise an instruction manual.

In an embodiment, the kit comprises reagents for performing multiplex PCR.

In an embodiment, the kit further comprises reagents for extracting total RNA from one or more biological samples. The biological samples can be PBMCs or biopsies.

The following examples illustrate embodiments of the invention and do not limit the scope of the invention.

### EXAMPLES

### Example 1

### Materials and Methods

### Study Design

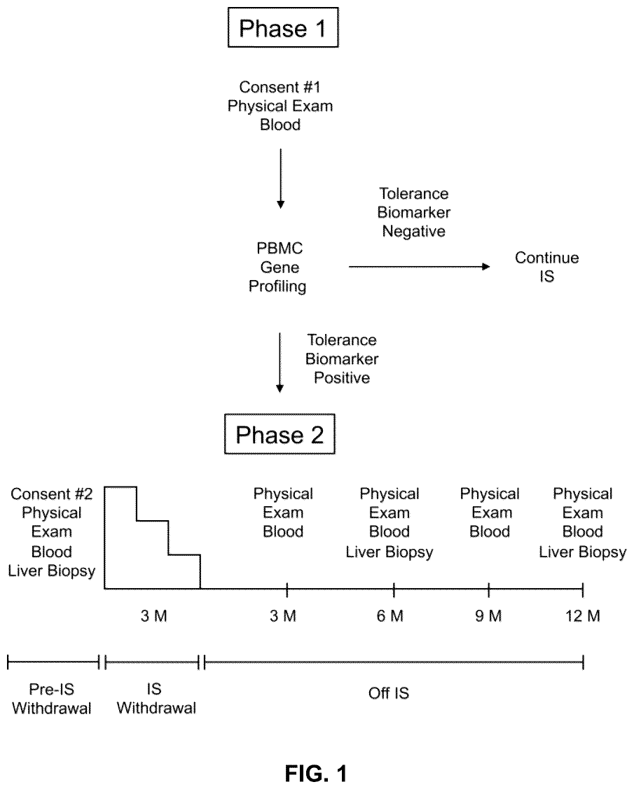

The Liver Immune Tolerance bioMarker Utilization Study (LITMUS, ClinicalTrials.gov NCT02541916) was a prospective observational single-centre, single-arm study conducted at the Toronto General Hospital, University Health Network (Toronto, Canada). LITMUS was approved by the Research Ethics Board of the University Health Network (14-8691). An independent data safety monitoring board monitored the trial. All patients provided written, informed consent. The inclusion and exclusion criteria for entry into the study are provided in Table 1. Twenty-three healthy living donors from the liver transplant program at the University Health Network who had normal liver biopsies were enrolled in the study and served as a control group for the gene expression analysis. FIG. 1 shows the design of the trial including timing of blood and liver biopsies for GeXP analysis. In Phase 1 of LITMUS, blood samples were collected from eligible patients for PBMC gene expression analysis. Patients who had the predefined gene expression ratio (FGL2/IFNγ 1) and a normal baseline liver biopsy were eligible to enter the IS withdrawal phase of the study (Phase 2). All cases eligible for Phase 2 were reviewed by an independent selection committee to confirm that there were no contraindications to IS withdrawal. In those patients who entered Phase 2 of the study, IS was withdrawn over 3 to 4 months in a stepwise fashion (25% reduction each month). Liver function tests were performed every 2 weeks during the withdrawal period, weekly for 1 month after cessation of IS, and then monthly for the next 11 months. Percutaneous liver biopsies and liver chemistry were performed at baseline, 6 months, and 1 year post-IS withdrawal. Although not formally part of the study, it was recommended patients undergo liver biopsy yearly for up to 5 years post-IS withdrawal.

TABLE 1
Inclusion and exclusion criteria

Inclusion criteria:
1) Be between 18 and 70 years of age
2) Be recipients of a hepatic allograft
3) Be a minimum of 3 months post-transplant
4) Have normal liver function tests for the 3 months prior to entry
including AST, ALT, ALP, bilirubin, and prothrombin time (INR)
5) Be free of rejection in the previous 3 months prior to enrolment
Patient exclusion criteria:
1) Patients under the age of 18 and over the age of 70
2) Patients who are positive for HIV
3) Patients who have detectable levels of HCV RNA or HBV DNA at the
time of enrolment
4) Patients who have a combined transplant and/or have been
re-transplanted
5) Patients taking IS for other diseases besides their liver transplant
6) Patients unable to give written informed consent in accordance
with research ethics board guidelines
HBV, hepatitis B virus; HCV, hepatitis C virus; INR, international normalized ratio.

### Definition of Rejection

Suspected rejection was diagnosed by disturbances in liver biochemistry including aspartate aminotransferase (AST), alanine aminotransferase (ALT), alkaline phosphatase (ALP), and bilirubin but all rejection episodes were confirmed by liver biopsy findings according to the Banff criteria [21]. Patients who had documented rejection were treated with a short course of oral steroids and reinstitution of IS.

### Definition of Operational Tolerance

Patients were classified as operationally tolerant as long as complete cessation of IS was maintained for a minimum of 12 months and no histologic evidence of rejection was observed.

### Study End Points

The primary aim of the study was the development of operational tolerance in patients who had the PMBC gene ratio (FGL2/IFNγ≥1). Secondary end points were mortality, graft loss, changes in adverse effects associated with cessation of IS and assessment of immune markers/gene expression in peripheral blood and liver allografts including RNA sequencing described below.

### Liver Biopsy Specimens

Percutaneous liver biopsies were performed under local anaesthesia using an 18-gauge Jamshidi needle. A 0.5-cm portion of the biopsy to be used for gene expression was stored in RNALater (Qiagen, Germantown, MA, USA) for 24 h at 4° C. and then transferred to −80° C. The remainder of the biopsy was used for histologic examination and was formalin-fixed and paraffin-embedded.

### Histologic Examination of Liver Biopsies

Haematoxylin-eosin- and Masson trichrome-stained sections were examined by two local pathologists who were blinded to all clinical and biological data. Biopsies were evaluated using the Banff criteria [21, 22]. For entry into Phase 2 of the study, patients were required to have a normal liver biopsy, defined as the absence of cellular, ductopenic, antibody-mediated, or other form of rejection; absence of active interface or lobular inflammation; absence of other active parenchymal or biliary injury; and fibrosis not more than Laennec stage 2.

### Isolation of Peripheral Blood Mononuclear Cells

Peripheral blood mononuclear cells (PBMC) were isolated from whole blood using Cell Preparation Tubes with sodium heparin (Becton, Dickinson and Company, Franklin Lakes, NJ, USA) following the manufacturer's instructions. PBMC were resuspended in RNAprotect cell reagent (Qiagen, Germantown, MA, USA) and stored at −80° C. for Multiplex RT-PCR and qPCR studies. PBMC were also cryopreserved in freezing media (10% DMSO in fetal calf serum) for immunophenotyping studies.

RNA Extraction from PBMC and Liver Biopsies

Total RNA was isolated from PBMC preserved in RNAprotect (Qiagen) using the PureLink RNA Mini Kit (Ambion, Austin, TX, USA) following the manufacturer's instructions. The final RNA precipitate was dissolved RNA Storage Solution (Ambion). RNA aliquots were stored at −80° C. for future use in GeXP multiplex reverse transcriptase polymerase chain reaction (RT-PCR), quantitative PCR (qPCR), and RNA sequencing (RNA-seq) studies.

Total RNA was extracted from liver biopsies preserved in RNALater. Specimens were thawed and transferred to a clean, RNase-free microcentrifuge tube. Tissue samples were incubated in lysis buffer (Ambion) with 2-mercaptoethanol for 10 min. Tissue samples were then homogenized using a nuclease-free disposable pellet pestle (Kimble Kontes, Vineland, NJ, USA) and processed using the PureLink RNA Mini Kit (Ambion).

### GeXP Multiplex RT-PCR

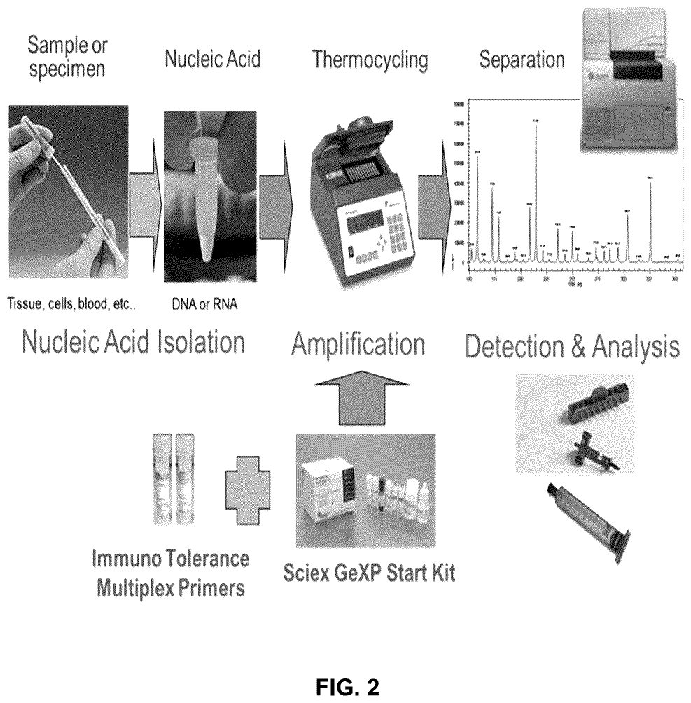

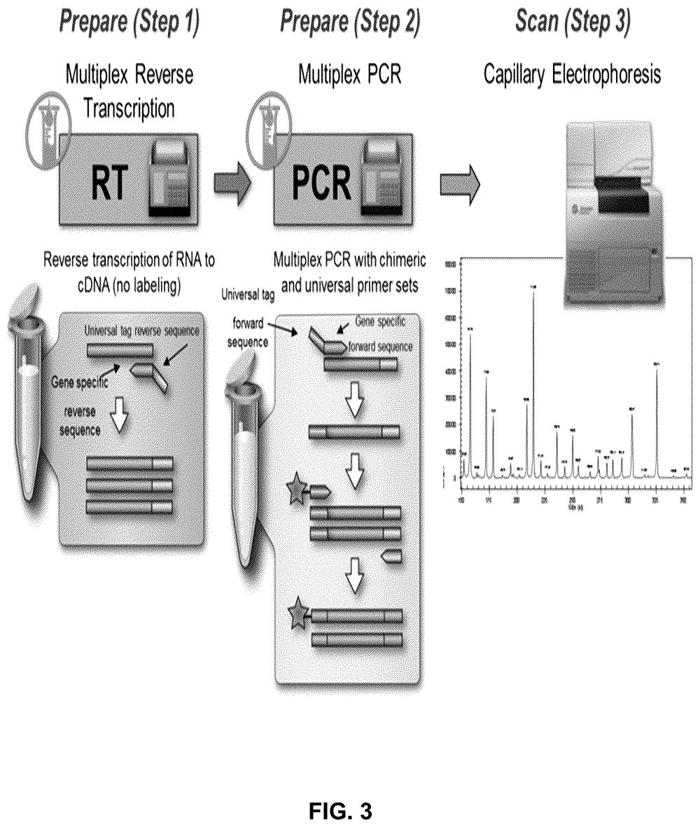

Thirteen genes were analysed in a single PCR using the GenomeLab GeXP Genetic Analysis System (SCIEX, Brea, CA, USA) (FIGS. 2-3). Eight target genes (Table 2) [FGL2, forkhead box P3 (FOXP3), transforming growth factor-β1 (TGFB1), lymphocyte activating-3 (LAG3), T-cell immunoreceptor with Ig and ITIM domains (TIGIT), interleukin-10 (IL10), IFNγ; and GZMB] were chosen for multiplexing based on previous studies [18, 19], and five housekeeping genes [hypoxanthine-guanine phosphoribosyltransferase (HPRT), TATA box binding protein (TBP), beta-2 microglobulin (B2M), cancer susceptibility candidate-3 (CASC3), and ezrin (EZR)] were used to normalize the data. Primers were designed to ensure that five nucleotides or more separated the length of each of the 13 amplicons in the gene panel, allowing for each gene amplicon to be characterized by a unique distinct peak on the electropherogram.

TABLE 2
Tolerance panel fragment size and accession numbers

| | Actual Size | Accession | Isoforms & variants |
|---|---|---|---|
| Gene | (w/tag) | Number | accession numbers |
| LAG3 | 163 | NM_002286.5 | XM_011520956.1 |
| TGFB1 | 169 | NM_000660.6 | XM_011527242.1 |
| FGL2 | 194 | NM_006682.2 | |
| FOXP3 | 210 | NM_014009.3 | NM_001114377.1, |
| | | | XM_017029566.1, |
| | | | XM_017029565.1, |
| | | | XM_006724533.2 |
| IFNγ | 250 | NM_000619.2 | |
| GZMB | 264 | NM_004131.5 | NM_001346011.1 |
| IL10 | 280 | NM_000572.2 | XM_011509506.1 |
| TIGIT | 368 | NM_173799.3 | |

Total RNA from PBMC and liver biopsies were processed using the GenomeLab GeXP Start Kit (SCIEX) following the manufacturer's protocols. RNA was first reverse-transcribed using the primer mixture and the reverse transcription reaction mixture from the GenomeLab GeXP Start Kit (SCIEX) and kanamycin resistance gene (kanR) RNA, which served as an internal control. A PCR mixture was then assembled using cDNA product from the reverse transcription step, forward primer mixture, and Thermo-Start DNA Polymerase. The forward primer mixture consisted of custom-designed gene-specific primers along with a fluorescent dye-labelled universal primer. PCR products were then separated by capillary electrophoresis using the GenomeLab GeXP Genetic Analysis System (SCIEX). Using the kanR signal as the reaction control for each well, the GeXP Quant Tool program normalized the fragment data to the kanR signals by dividing the peak area of each gene peak by the peak area of kanR. Gene expression values were then normalized to the house keeping genes and subsequently expressed as a ratio compared with gene expression in PBMC or liver tissue obtained during surgery from live liver donors who served as healthy controls.

### Quantitative PCR

To validate the GeXP results, qPCR was performed. Gene expression of eight genes of interest (FGL2, FOXP3, TGFB1, LAG3, TIGIT, IL10, IFNγ; and GZMB) and two housekeeping genes (HPRT and TBP) was measured by real-time qPCR (qPCR) using the LightCycler 480 SYBR Green I Master (Roche Applied Science, Indianapolis, IN, USA) and LightCycler 480 Real-Time PCR Machine (Roche Diagnostics, Rotkreuz, Switzerland). The primers for qPR amplification of the target and housekeeping genes are shown in Table 2. Total RNA was reverse-transcribed into complementary DNA (cDNA) using the SuperScript IV First Strand Synthesis System (Invitrogen, Waltham, MA, USA). Real-time qPCR was then performed in a 10 μl volume of 2× Master Mix (5 μl), 5 μM primer mixture (1 μl), PCR-grade water (2 μl), and cDNA diluted 1:7 (2 μl). Samples were run in triplicate and all results were normalized to HPRT. The 2-ΔΔ^{Ct} method was used to calculate gene expression of target genes relative to housekeeping gene HPRT [23].

TABLE 2
Primers for qPCR

| | | SEQ |
|---|---|---|
| | | ID |
| Name | Sequence | No |
| Forward_FGL2 | AAAGTGTCCCAGCCAAGAACA | 1 |
| Reverse_FGL2 | CTCTGTAGGTCTCACTGCTTCTTTTG | 2 |
| Forward_ | TGATACGTGACAGTTTCCCACAA | 3 |
| FOXP3 | | |
| Reverse_ | TTGGGCATCGGGTCCTT | 4 |
| FOXP3 | | |
| Forward_TGF- | CCAGCGACTCGCCAGAGT | 5 |
| β1 | | |
| Reverse_TGF- | GGCGAAAGCCCTCAATTTC | 6 |
| β1 | | |
| Forward_TIGIT | GATGGGACGTACACTGGGAGAA | 7 |
| Reverse_TIGIT | TCCAAGCAATGGAATCTGGAA | 8 |
| Forward_IL-10 | GAGAACAGCTGCACCCACTTC | 9 |
| Reverse_IL-10 | CAGCTGATCCTTCATTTGAAAGAA | 10 |
| Forward_LAG3 | CTGGGACCTACACCTGCCATA | 11 |
| Reverse_LAG3 | AGGATTTGGGAGTCACTGTGATG | 12 |
| Forward_GZMB | AAACAACAGCAGCTCCAACCA | 13 |
| Reverse_GZMB | CGATGATCTCCCCTGCATCT | 14 |
| Forward_INF-γ | TCAATAGCAACAAAAAGAAACGAGAT | 15 |
| Reverse_INF-γ | CATGTATTGCTTTGCGTTGGA | 16 |
| Forward_HRPT | TGCTGAGGATTTGGAAAGGG | 17 |
| Reverse_HRPT | ACAGAGGGCTACAATGTGATG | 18 |
| Forward_TBP | GAGAGTTCTGGGATTGTACCG | 19 |
| Reverse_TBP | ATCCTCATGATTACCGCAGC | 20 |

### Mass Cytometry

Peripheral blood immune cell subsets were characterized with a 36-parameter mass cytometry panel (Table 3) based on the immune monitoring flow cytometry markers used in the ONE Study of immunoregulatory cell therapy in renal transplantation [24]. Cryopreserved PBMC were recovered and stained with cis-platinum and a DNA intercalator to distinguish live cells from debris. After staining with antibodies and washing, data were acquired on a CyTOF 2 mass cytometer (Fluidigm, South San Francisco, CA, USA) and analysed with conventional gating using Cytobank software (Cytobank, Santa Clara, CA, USA).

TABLE 3
List of antibodies in mass cytometry panel

| Number | Isotope | Target | Clone | Vendor |
|---|---|---|---|---|
| 1 | 89Y | CD45 | HI30 | Fluidigm |
| 2 | 115In | CD57 | HCD57 | Biolegend |
| 3 | 141Pr | HLA-DR | L243 | Biolegend |
| 4 | 142Nd | CD45-RO | UCHL1 | Biolegend |
| 5 | 143Nd | CD11c | Bu15 | Biolegend |
| 6 | 145Nd | CD4 | SK3 | Biolegend |
| 7 | 146Nd | CD8a | RPA-T8 | Biolegend |
| 8 | 147Sm | CD31 | WM59 | Biolegend |
| 9 | 148Nd | IgD | IA6-2 | Biolegend |
| 10 | 149Sm | FoxP3 | 236A/E7 | Thermo Fisher |
| 11 | 150Nd | CD69 | FN50 | Biolegend |
| 12 | 151Eu | CD123 | 6H6 | Biolegend |
| 13 | 152Sm | Tbet | 4B10 | BD |
| | | | | Biosciences |
| 14 | 153Eu | CD45RA | HI100 | Biolegend |
| 15 | 154Sm | CD19 | HIB19 | Thermo Fisher |
| 16 | 156Gd | CD28 | CD28.2 | Biolegend |
| 17 | 158Gd | CD33 | WM53 | Biolegend |
| 18 | 159Tb | Helios | 22F6 | Biolegend |
| 19 | 160Gd | CD14 | M5E2 | Biolegend |
| 20 | 161Dy | CD25 | M-A251 | BD |
| | | | | Biosciences |
| 21 | 162Dy | CD38 | HIT2 | Biolegend |
| 22 | 163Dy | CD127 | eBioRDR5 | Thermo Fisher |
| 23 | 164Dy | CD39 | A1 | Biolegend |
| 24 | 165Ho | IgM | MFM-88 | Biolegend |
| 25 | 166Er | Rorγt | 7C9 | Biolegend |
| 26 | 167Er | CD27 | O323 | Biolegend |
| 27 | 168Er | TIGIT | A15153G | Biolegend |
| 28 | 169Tm | CD24 | ML5 | Biolegend |
| 29 | 170Er | CD3 | UCHT1 | Thermo Fisher |
| 30 | 171Yb | PD-L1 | 29E.2A3 | Biolegend |
| 31 | 172Yb | TCRγδ | 5A6.E9 | Biolegend |
| 32 | 173Yb | CD294 | BM16 | Biolegend |
| 33 | 174Yb | CD56 | NCAM16.2 | BD |
| | | | | Biosciences |
| 34 | 175Lu | CD279 (PD1) | EH12.2H7 | Biolegend |
| 35 | 176Yb | CCR7 | G043H7 | Biolegend |
| 36 | 209Bi | CD16 | 3G8 | Fluidigm |

### Liver Immunofluorescence

Intrahepatic immunophenotyping was performed as previously described [17, 25, 26]. Briefly, the portal infiltrate size was determined by encircling portal infiltrates along the limiting plate and excluding the lumen of veins, arteries, and bile ducts. The intrahepatic infiltration of CD4⁺CD8⁻FOXP3⁻ (CD4⁺), CD8⁺CD4⁻FOXP3⁻ (CD8⁺), CD4⁺CD8⁻FOXP3⁺ (CD4⁺FOXP3⁺ Tregs), and CD8⁺CD4⁻FOXP3⁺ (CD8⁺FOXP3⁺) cells was quantified. In the current study 95% of portal FOXP3⁺ cells were CD4⁺, and only 5% were potentially activated CD8⁺FOXP3⁺ T effector cells excluding a significant contamination of activated T effector cells in the pool of CD4⁺FOXP3⁺ Tregs. The immunohistological Treg detection in human FFPE tissue sections was recently validated by flow cytometric and epigenetic analysis [17, 25].

### RNA-Seq and Analysis

RNA previously isolated from baseline liver biopsies for GeXP studies was used for RNA-seq, which was performed at The Centre for Applied Genomics (Toronto, Canada). Briefly, RNA quality was evaluated using a Bioanalyzer 2100 (Agilent, Santa Clara, CA, USA) and samples with an RNA integrity number (RIN) >7 were submitted for sequencing. RNA library preparation was performed with the NEBNext Ultra II Directional RNA Library Prep kit (New England Biolabs, Ipswich, MA, USA). Libraries were sequenced on a High Throughput Run Mode flowcell with V4 sequencing chemistry on a HiSeq 2500 (Illumina, San Diego, CA, USA) platform following the manufacturer's recommended protocol. Generated sequence fragments were aligned to the reference genome (GRCh38, Gencode annotations, Release 356) using the STAR aligner, v.2.6.0c. The filtered STAR alignments were processed to extract raw read counts for genes using Htseq-count v.0.6.1p2. Only uniquely mapping reads are counted. Two-condition differential gene expression analysis was performed with DESeq2 v.1.26.0s, using R v.3.6.1. Initial minimal filtering of 10 read counts per gene for at least three samples was applied to the data set. A cut-off of P adjusted (P_{adj}) <0.05 by the Benjamini-Hochberg method was used to identify genes with differential expression between tolerant and non-tolerant groups.

### Statistics

Continuous variables were analysed with either a t-test for normally distributed data or a Mann-Whitney U test for non-normally distributed data. Categorical variables were analysed with a Fisher's exact test. Analysis of gene expression and liver immunohistochemistry was performed with Kruskal-Wallis test followed by Dunn's multiple comparisons post hoc test. Statistical analyses were performed using the Graphpad Prism version 8 software package (Graphpad Software, La Jolla, CA, USA). P-values of ≤0.05 were considered statistically significant.

### Example 2

### Development of a Human GeXP Gene Expression Assay

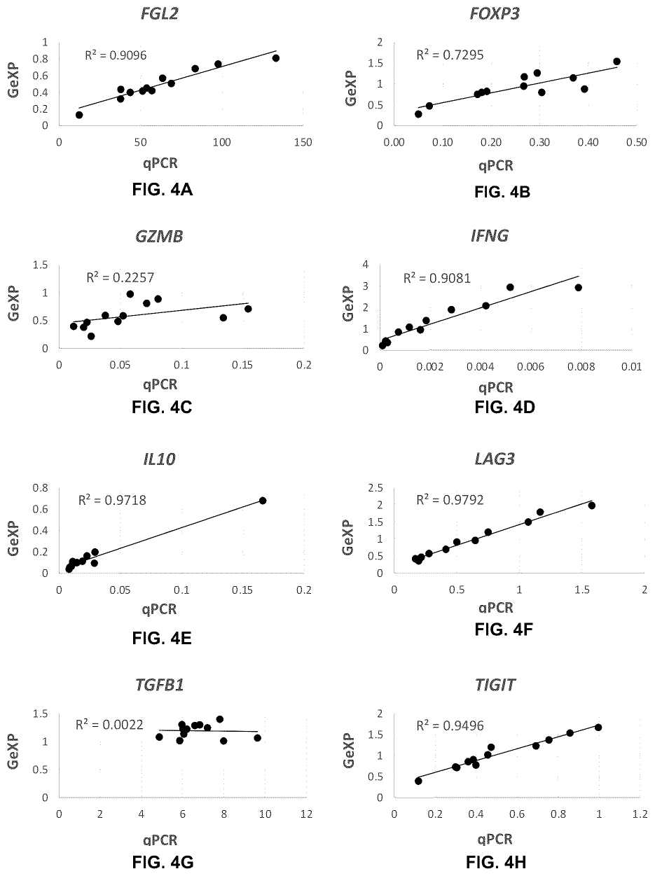

A human GeXP assay was developed to quantify expression of eight target genes (FGL2, FOXP3, TGFB1, LAG3, TIGIT, IL10, IFNγ, and GZMB) previously identified to be predictive of tolerance in preclinical studies [18, 19]. Comparison of gene expression using this GeXP assay with real-time qPCR showed a high degree of correlation with R-square values >0.72 for six of the genes (FGL2, FOXP3, IFNγ, IL10, LAG3, TIGIT) (FIG. 4).

### Example 3

### Patients and Clinical Outcomes

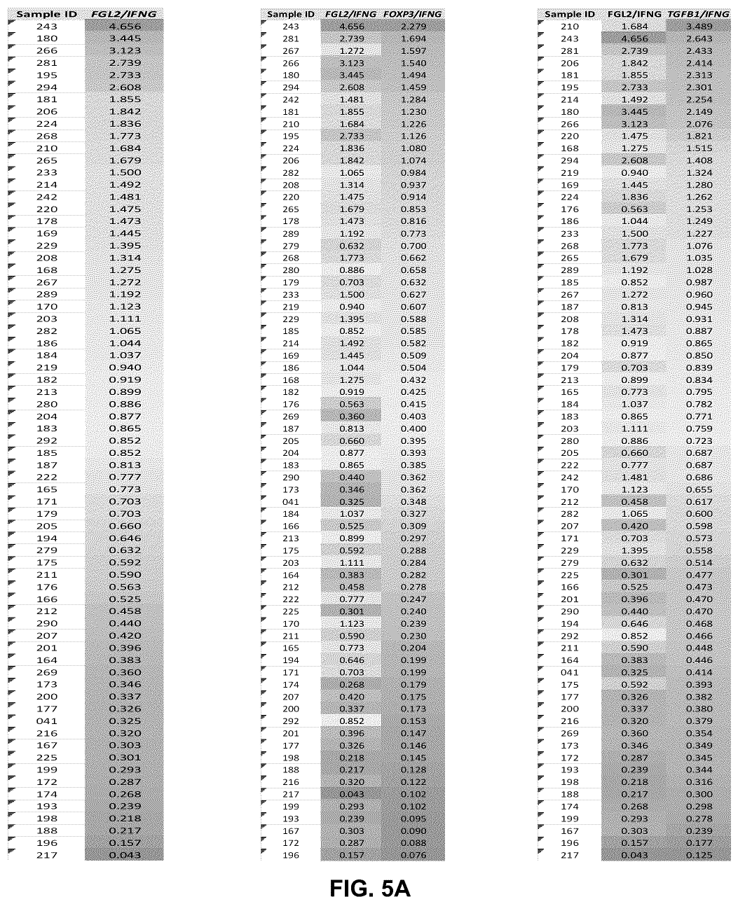

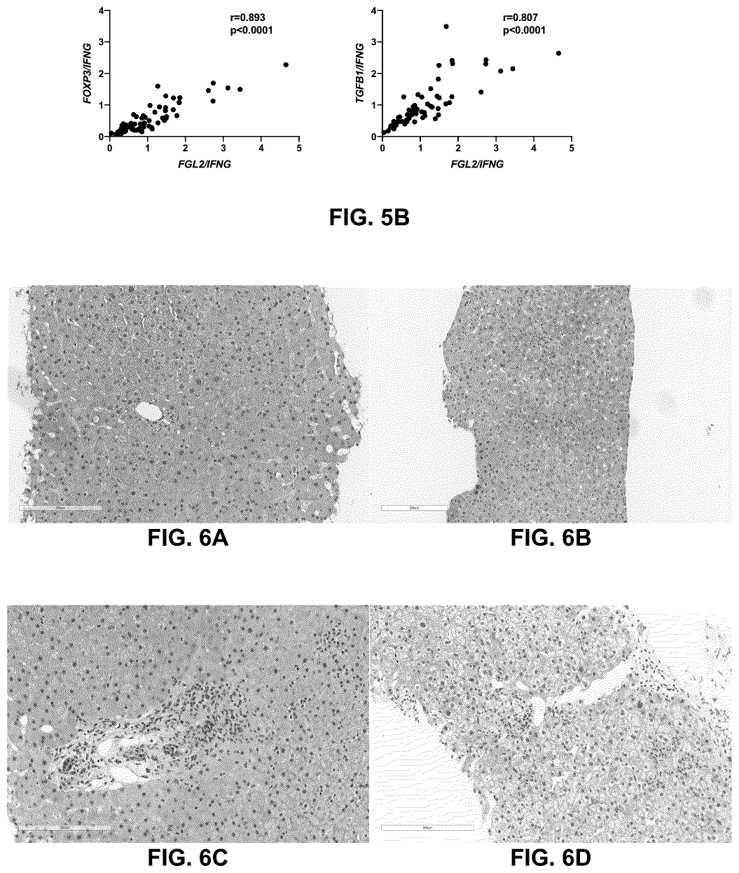

Sixty-nine liver transplant recipients who were a minimum of 6 months post-liver transplant and had no documented rejection episodes in the previous 3 months were enrolled over a 54-month period from May 2015 to November 2019. Patients with autoimmune liver disease and non-active (no viral replication) viral hepatitis B and C were eligible for inclusion in the trial. After patients entered the study, their PBMC gene expression was determined using the custom GeXP assay. Similar to the murine preclinical studies of tolerance, the eight genes in the assay were expressed as increased or decreased in relation to normal healthy controls and as the ratio of anti-inflammatory to pro-inflammatory genes (e.g. FGL2/IFNγ, IL10/IFNγ, TIGIT/IFNγ, and TGFB1/IFNγ). An elevated PBMC ratio of FGL2/IFNγ, which was predictive of tolerance in preclinical models of tolerance, was used to as a putative tolerance biomarker in LITMUS. Overall, 28 of the 69 patients were positive for the tolerance biomarker (FGL2/IFNγ≥1). Interestingly, the order of 69 patients remained relatively unchanged when patients were sorted by the ratio of FGL2/IFNγ, FOXP31 IFNγ, or TGFB1/IFNγ, suggesting that there may be coordinated expression of other immunoregulatory genes (FIG. 5). Table 4 shows the clinical characteristics of the entire cohort and patients who were positive and negative for the tolerance biomarker. Compared with patients in the negative tolerance biomarker group, patients in the positive biomarker group had a higher percentage mycophenolate mofetil (MMF) use and a lower percentage of hepatitis B virus (HBV) as an indication for transplantation (Table 4).

TABLE 4
Clinical characteristics of the entire trial cohort, patient
who were positive for the tolerance biomarker, and patients
who were negative for the tolerance biomarker.

| | | Tolerance | Tolerance | |
|---|---|---|---|---|
| | Total | biomarker | biomarker | |
| | cohort | positive | negative | |
| | (n = 69) | (n = 28) | (n = 41) | P-value |
| Age (years) | 56 ± 15 | 55 ± 16 | 57 ± 14 | 0.51 |
| Sex (% male) | 61 | 57 | 63 | 0.62 |
| Diagnosis (number | | | | |
| of patients) | | | | |
| HCV cirrhosis | 18 | 9 | 9 | 0.41 |
| HBV cirrhosis | 10 | 1 | 9 | 0.04 |
| Alcoholic cirrhosis | 12 | 3 | 9 | 0.34 |
| Autoimmune | 5 | 2 | 3 | 0.99 |
| FHF | 5 | 4 | 1 | 0.15 |
| NASH | 6 | 4 | 2 | 0.21 |
| Other | 13 | 5 | 8 | 0.99 |
| Time from transplant | 111 ± 74 | 110 ± 69 | 111 ± 76 | 0.98 |
| to trial enrolment | | | | |
| (months) | | | | |
| No history of | 70 | 71 | 68 | 0.78 |
| rejection post-liver | | | | |
| transplant (%) | | | | |
| IS regimen (%) | | | | |
| Tacrolimus | 68 | 68 | 68 | 0.99 |
| Cyclosporine | 32 | 32 | 32 | 0.99 |
| MMF | 9 | 18 | 2 | 0.04 |
| Donor age (years) | 45 ± 18 | 44 ± 15 | 45 ± 18 | 0.83 |
| Type of | | | | 0.99 |
| transplantation (%) | | | | |
| Deceased donor | 83 | 82 | 83 | |
| Living donor | 17 | 18 | 17 | |
| Laboratory values at | | | | |
| enrolment | | | | |
| Creatinine | 99 ± 34 | 99 ± 25 | 99 ± 40 | 0.96 |
| AST | 25 ± 8 | 26 ± 8 | 24 ± 8 | 0.33 |
| ALT | 24 ± 13 | 27 ± 14 | 22 ± 12 | 0.16 |
| ALP | 104 ± 40 | 108 ± 45 | 102 ± 37 | 0.58 |
| Bilirubin | 14 ± 11 | 11.9 ± 5.6 | 14.8 ± 12.9 | 0.27 |
| INR | 1.1 ± 0.4 | 1.2 ± 0.6 | 1.1 ± 0.3 | 0.67 |

Data are expressed at mean ± SD. FHF, fulminant hepatic failure; HBV, hepatitis B virus; HCV, hepatitis C virus; INR, international normalized ratio; MMF, mycophenylate mofetil; NASH, non-alcoholic steatohepatitis.

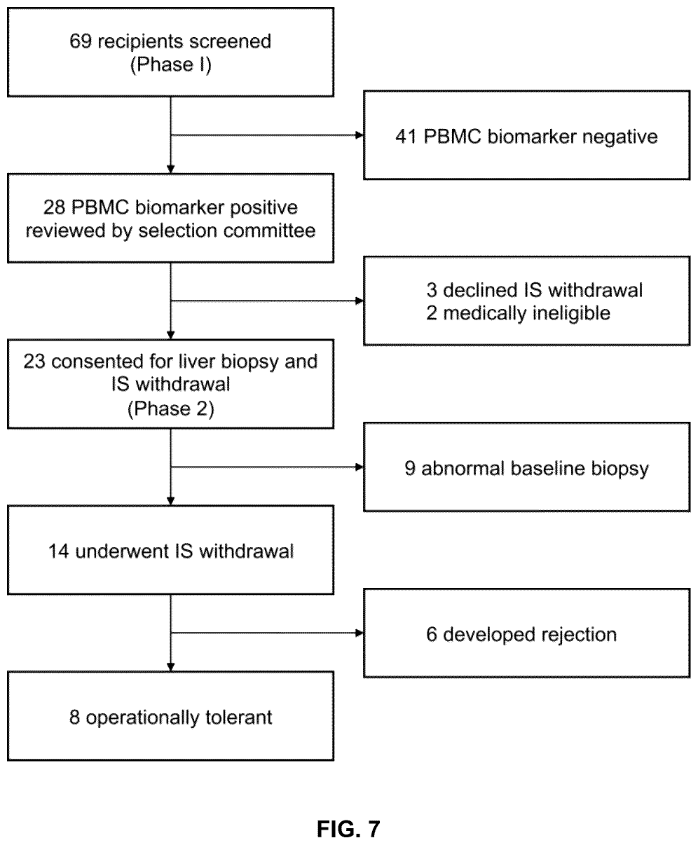

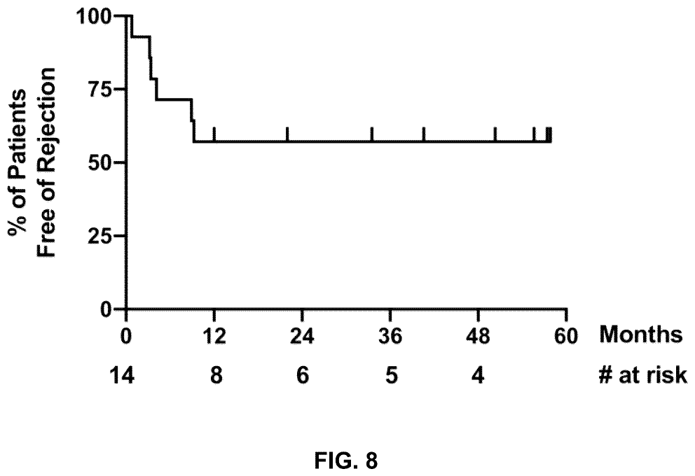

Of the 28 eligible patients, 23 entered the IS withdrawal phase of LITMUS (Phase 2), three patients refused entry, and two patients were deemed medically ineligible due to pre-existing medical conditions (one patient had polycythaemia vera and preleukemia, and another patient had a history of hepatic artery and portal vein thromboses and was on systemic anticoagulation). Of the 23 patients, nine had evidence of recurrent or de novo autoimmune liver disease or subclinical cell-mediated rejection on their liver biopsies (FIG. 6) and thus were excluded from the withdrawal of IS phase of the study. Fourteen patients had normal liver biopsies and entered the IS withdrawal phase of the study. No patients with a diagnosis of autoimmune liver disease (autoimmune hepatitis, primary biliary cholangitis, or primary sclerosing cholangitis) had the tolerant profile and a normal liver biopsy. Of the patients who entered the IS withdrawal phase of the study (Phase 2), eight patients have been successfully weaned off IS and are operationally tolerant (IS-free for a range of 12-57 months). Six patients developed rejection which occurred either during IS weaning (n=3) or post-IS withdrawal (n=3) (FIGS. 7 and 8). All of the rejection episodes were mild (Banff score less than 5) and were easily reversed with a short course of steroids and reinstitution of IS. No patients suffered graft loss or any long-term side effects and their liver chemistry returned to normal following treatment.

Table 5 shows baseline characteristics of the operationally tolerant patients versus non-tolerant patients who either developed rejection or had abnormal liver biopsies. Although there was no difference in age between the two groups, two of the patients who developed tolerance were less than 30 years of age. Compared with non-tolerant patients, operationally tolerant patients had a longer time from transplant to enrolment and a lower baseline ALT. Choice and drug levels of calcineurin inhibitor at entry into the study (pre-IS withdrawal) were not statistically different between tolerant and non-tolerant patients. Although MMF usage was increased in the high biomarker ratio patients, its use was not different between tolerant and non-tolerant patients (Table 5). The ability of other gene ratios (FOXP3I IFNγ and TGFBI IFNγ) to identify tolerant patients was also examined. Using these gene ratios, less patients would have been identified as having a positive ratio (ratio value ≥1) and less tolerant patients would have been identified than with FGL2/IFNγ ratio (Table 6).

TABLE 5
Clinical characteristics of operationally
tolerant patients and non-tolerant patients

| | Operationally tolerant | Non-tolerant | |
|---|---|---|---|
| | (n = 8) | (n = 15) | P-value |
| Age (years) | 56 ± 19 | 55 ± 16 | 0.93 |
| Sex (% males) | 63 | 60 | 0.99 |
| Diagnosis (number | | | |
| of patients) | | | |
| HCV cirrhosis | 3 | 5 | 0.99 |
| Alcoholic cirrhosis | 0 | 2 | 0.53 |
| Autoimmune | 0 | 2 | 0.53 |
| FHF | 1 | 1 | 0.99 |
| NASH | 1 | 3 | 0.99 |
| Other | 3 | 2 | 0.29 |
| Time from transplant | 158 ± 83 | 91 ± 51 | 0.03 |
| to trial enrolment | | | |
| (months) | | | |
| Free of rejection prior | 75 | 68 | 0.99 |
| to enrolment (%) | | | |
| IS regimen prior to | | | |
| withdrawal (%) | | | |
| Tacrolimus | 50 | 73 | 0.37 |
| Cyclosporine | 50 | 27 | 0.37 |
| MMF | 13 | 20 | 0.99 |
| CNI drug levels prior | | | |
| to IS withdrawal (ng/ml) | | | |
| Tacrolimus (trough level) | 5.8 ± 0.5 | 5.9 ± 1.5 | 0.68 |
| Cyclosporine (C2 level) | 354 ± 245 | 278 ± 133 | 0.89 |
| Donor age (years) | 48 ± 7 | 46 ± 9 | 0.82 |
| Type of | | | 0.99 |
| transplantation (%) | | | |
| Deceased donor | 88 | 80 | |
| Living donor | 12 | 20 | |
| Laboratory values at | | | |
| enrolment | | | |
| Creatinine | 107 ± 36 | 94 ± 18 | 0.27 |
| AST | 22 ± 4 | 29 ± 10 | 0.07 |
| ALT | 19 ± 6 | 33 ± 17 | 0.05 |
| ALP | 99 ± 33 | 110 ± 53 | 0.61 |
| Bilirubin | 12.6 ± 5 | 10.6 ± 10.6 | 0.33 |
| INR | 1 ± 0 | 1 ± 0.1 | 0.43 |

Data are expressed at mean ± SD. CNI, calcineurin inhibitor; FHF, fulminant hepatic failure; HCV, hepatitis C virus; INR, international normalized ratio; NASH, non-alcoholic steatohepatitis.
Patients with either unsuccessful IS withdrawal (n = 6) or abnormal baseline liver biopsy (n = 9).

TABLE 6
Results using different PBMC gene ratios to identify tolerant patients.

| | Patients | | | | Declined | | | |
|---|---|---|---|---|---|---|---|---|
| Gene | with | | Non- | Abnormal | IS | Medically | Unknown | % |
| Ratio | Ratio ≥1 | Tol | Tol | Biopsy | Withdrawal | Ineligible | Outcome ^{A} | Tol^{B} |
| FGL2/ | 28 | 8 | 6 | 9 | 3 | 2 | 0 | 57% |
| IFNγ | | | | | | | | |
| FOXP3/ | 12 | 4 | 3 | 3 | 1 | 1 | 0 | 57% |
| IFNγ | | | | | | | | |
| TGFB/ | 21 | 5 | 3 | 7 | 2 | 2 | 2 | 63% |
| IFNγ | | | | | | | | |

^{A} Patients who did not enter Phase 2 of LITMUS (n = 28 patients) as their FGL2/IFNγ <1and their outcome is unknown
^{B}Calculated as ratio of Tolerant/(Tolerant + Non-Tolerant)
IS, immunosuppression; Non-Tol, non-tolerant; PBMC, peripheral blood mononuclear cells; Tol, tolerant.

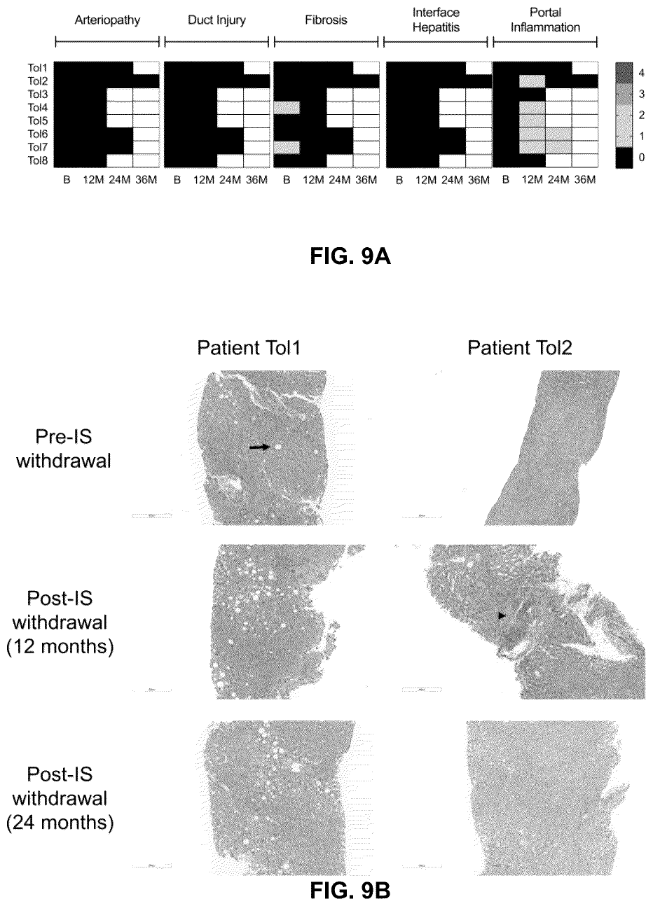

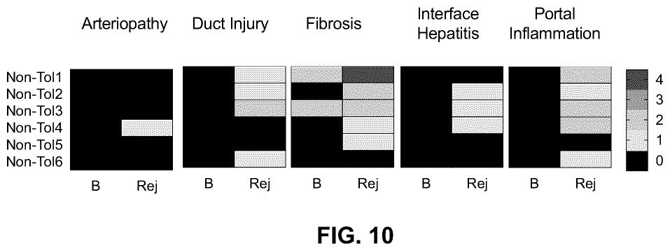

Protocol liver biopsies were performed pre- and post-IS withdrawal. Biopsies were examined especially for the presence of interface hepatitis, arteriopathy, bile duct loss, and fibrosis which have been reported to be associated with inability to wean off IS [22]. Results from a detailed analysis of biopsies from tolerant patients are shown in FIG. 9A. Pre-IS withdrawal, two of eight of the tolerant patients had mild fibrosis (Grade 1) without additional abnormalities, and six patients had no significant abnormalities. Post-IS withdrawal, there was no evidence of arteriopathy, fibrosis progression, interface hepatitis, or bile duct injury/loss in any of the biopsies from tolerant patients. However, five of the tolerant patients developed mild portal inflammation. FIG. 9B shows pre- and post-IS withdrawal biopsies from a patient who had no significant change in liver pathology post-IS withdrawal (Patient Tol1) and biopsies from another patient who developed mild portal infiltrates, which resolved on a subsequent biopsy (Patient Tol2). At the time of the diagnosis of rejection, liver biopsies from the six non-tolerant patients had evidence of rejection with mild to moderate portal and/or interface hepatitis (n=5), arteriopathy (n=1), bile duct injury (n=4), and increased fibrosis (n=4) (FIG. 10).

To determine if withdrawal of IS had clinical benefit, patients were monitored for effect on renal function, hypertension, diabetes, cancer, and death at 1 year post-IS withdrawal. Patients who were maintained on IS (PBMC tolerance biomarker negative) served as a control group. Patients successfully weaned off IS had numerical improvements in the incidence and severity of renal dysfunction and no new hypertension, diabetes, cancer, or death, but none of these reached statistical significance (Table 7).

TABLE 7
Development of transplant complications in operationally tolerant patients
and tolerance biomarker negative liver transplant recipients

| | Operationally | Tolerance biomarker | |
|---|---|---|---|
| | tolerant | negative | |
| | (n = 8) | (n = 41) | P-value |
| Renal function (change | −10.1 ± 15.1 | +0.6 ± 15.3 | 0.075 |
| from baseline; μmol/L) | | | |
| Hypertension | 0 (0%) | 3 (7.3%) | 0.9 |
| (new diagnosis) | | | |
| Diabetes | 0 (0%) | 1 (2.4%) | 0.9 |
| (new diagnosis) | | | |
| Malignancy | 0 (0%) | 1 (24%) | 0.9 |
| (new diagnosis) | | | |
| Death | 0 (0%) | 2 (4.9%) | 0.9 |

Data are presented as mean ± SD or number (percent). Change in clinical parameters since time of enrolment into trial (Phase 1).

### Example 4

Operationally Tolerant Patients have an Increase in Peripheral Blood Treg Post-IS Withdrawal

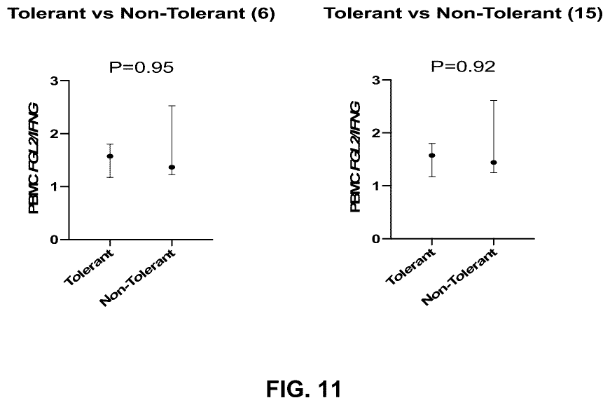

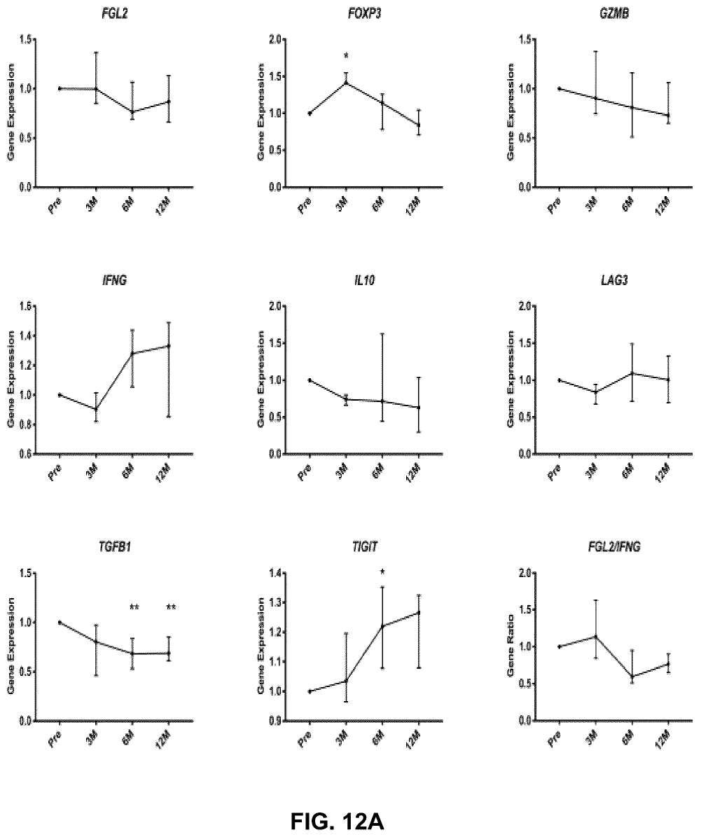

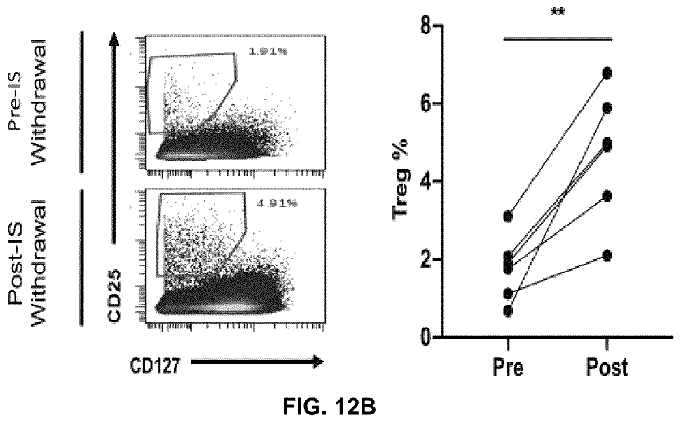

Further analysis of PBMC GeXP gene expression was performed to gain insights into mechanisms of tolerance. At baseline (prior to IS withdrawal), there were no differences in the gene panel and the FGL2I IFNγ gene ratio between operationally tolerant patients and non-tolerant patients (FIG. 11). In operationally tolerant patients, serial PBMC profiling revealed an early (3 months) increase in FOXP3 gene expression, consistent with an expansion of Tregs in the peripheral blood (FIG. 12A). There was also a significant increase in TIGIT (9 months) and decrease in TGFB1 (9 and 12 months) post-IS withdrawal (FIG. 12A).

In order to confirm GeXP gene changes, mass cytometry was performed on PBMC from operationally tolerant patients. These studies demonstrated a greater than 2.5-fold expansion of Tregs as a percentage of CD4⁺ cells post-IS withdrawal (1.78% vs. 4.72%, P<0.01) (FIG. 12B).

### Example 5

Operationally Tolerant Patients have an Elevated Baseline Liver FOXP3/IFNγ Gene Ratio and an Accumulation of Portal Tregs Post-IS Withdrawal

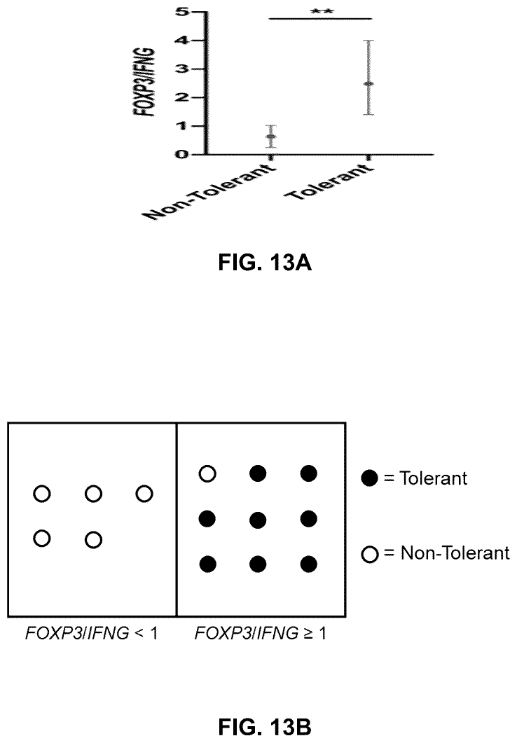

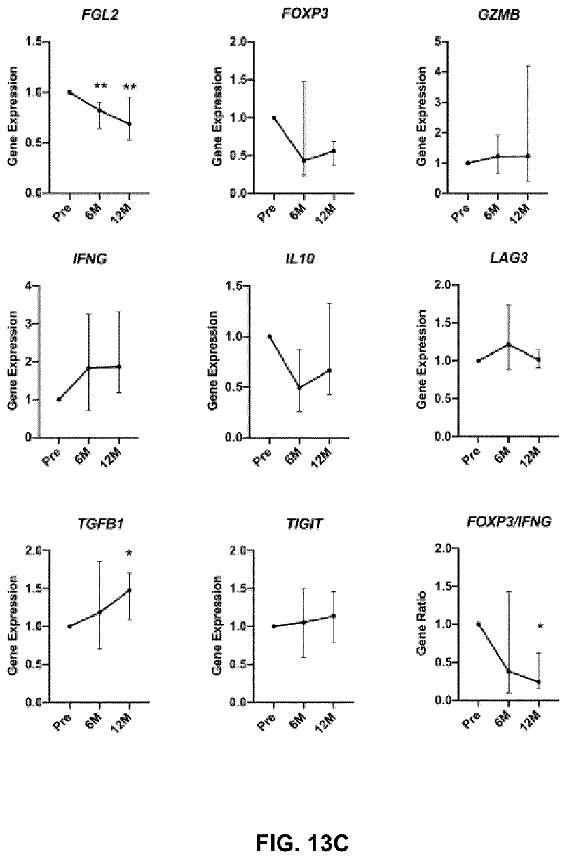

Gene expression from liver biopsies were also profiled with the GeXP assay to identify intrahepatic genes associated with tolerance. In contrast to baseline PBMC gene expression, there was a significant difference between baseline liver gene expression between operationally tolerant patients and patients who developed rejection (non-tolerant) with a higher intrahepatic FOXP3/IFNγ gene ratio in the tolerant patients (FIG. 13A). Furthermore, at a FOXP3/IFNγ ratio cut-off of 1, only one patient (17%) in the non-tolerant group had an elevated ratio, whereas all eight patients (100%) in the tolerant group had an elevated ratio (FIG. 13B). This result suggests that a higher ratio of intrahepatic Tregs to inflammatory cells at baseline may serve to identify tolerant liver transplant recipients. In the eight patients who achieved tolerance, profiling of liver gene expression over time revealed a decrease in this FOXP3/IFNγ ratio, consistent with inflammatory changes in the liver post-IS withdrawal (FIG. 13C). Over time there was also an increase in TGFB1 and a decrease in FGL2 intrahepatic gene expression in operationally tolerant patients (FIG. 13C).

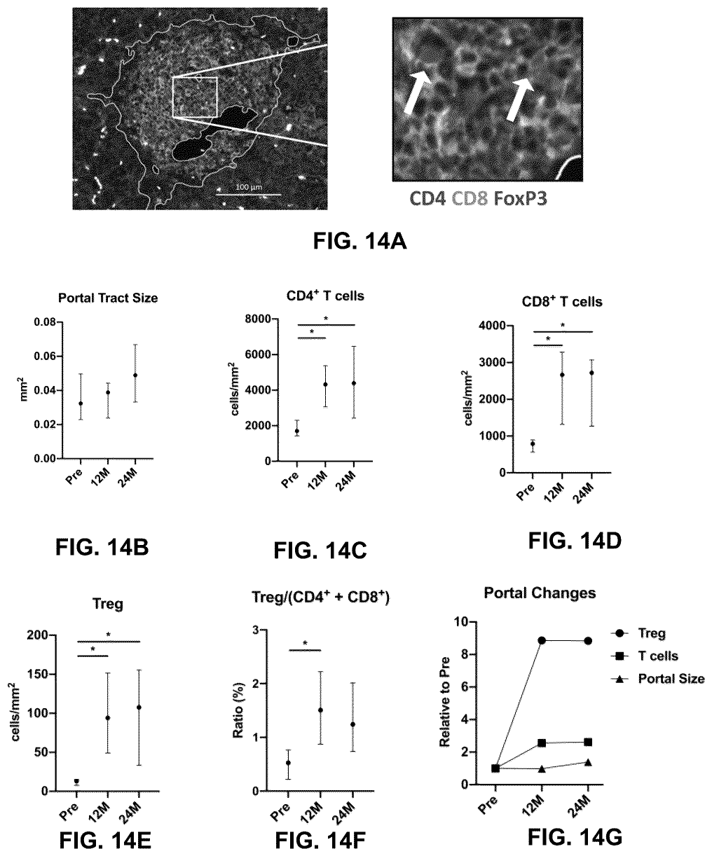

Immunofluorescence was performed on liver biopsies to delineate CD4⁺ T cells, CD8⁺ T cells, and FOXP3⁺ Tregs in operationally tolerant patients (FIG. 14A). After the complete withdrawal of IS, the size of portal tracts exhibited a non-significant trend to an approximately 50% expansion (FIGS. 14B and G). T cells as well as Tregs accumulated in the portal tracts after the weaning (FIGS. 14C-E). The lobular T-cell infiltration was persistently low and too variable for a systematic analysis. While both CD4⁺ and CD8⁺ T cells increased over time, Tregs accumulated disproportionately in the portal tracts compared to total T-cell numbers, as exemplified by an increasing ratio of Tregs to total CD4⁺ and CD8⁺ T cells (FIGS. 14F and G). The increase in portal Treg seen at 1 year persisted out to 2 years post-IS withdrawal (FIGS. 14E and G).

### Example 6

Operationally Tolerant Patients have Higher Baseline Liver Expression of SELE and Lower Expression of Genes Associated with Inflammatory Responses

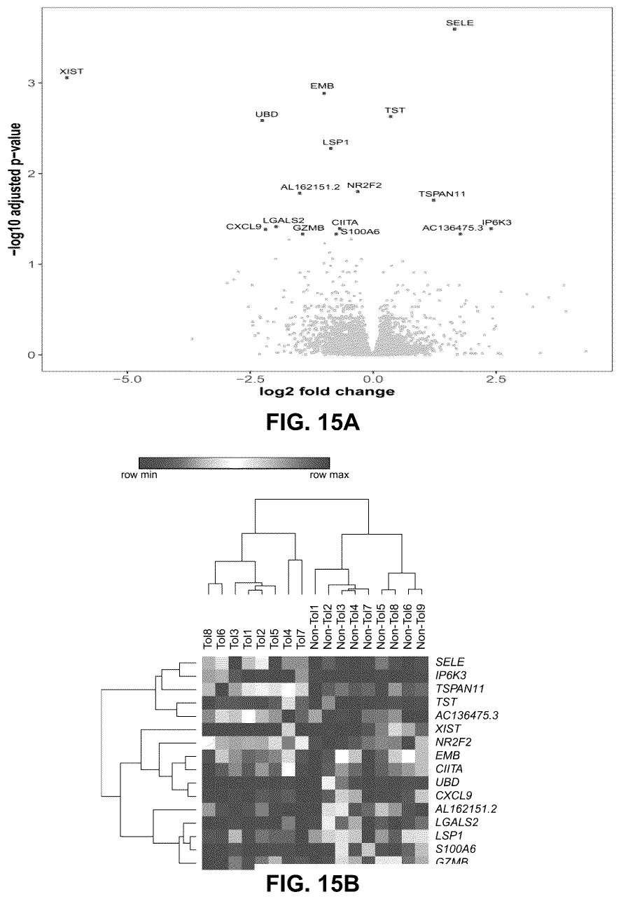

To gain further insights into mechanisms of tolerance induction, pre-IS withdrawal liver biopsies from patients who achieved operational tolerance and who were non-tolerant (abnormal biopsies at baseline or developed rejection during IS withdrawal) were analysed with RNA-seq. Overall, there were 16 genes that were differentially expressed between the two groups with five genes upregulated and 11 genes downregulated (Table 8). FIG. 15A shows a volcano plot and FIG. 15B shows a heatmap of these differentially expressed genes. The gene for E-selectin (SELE) was the most significantly upregulated gene in the tolerant group versus the non-tolerant group (P_{adj}=0.00026; log 2 fold change=1.67). Additional upregulated genes included tetraspanin 11 (TSPAN11), inositol hexakisphosphate kinase 6 (IP6K3), thiosulfate sulfurtransferase (TST), AC162151.2 (pseudogene), and AC136475.3 (long non-coding RNA). Genes that were reduced in the tolerant versus the non-tolerant group were associated with inflammatory responses including ubiquitin D (UBD), lymphocyte-specific protein-1 (LSP1), class II major histocompatibility complex transactivator (CIITA), C-X-C motif chemokine ligand 9 (CXCL9), and GZMB. The most significantly downregulated gene was X-inactive specific transcript (XIST). The significance of this is presently unknown but may be due to a mismatch in the sex of the liver donors between the two groups (two female liver donors in the tolerant group and five female donors in the non-tolerant group).

TABLE 7
Genes differentially expressed in baseline liver biopsies
(pre-immunosuppression withdrawal) between tolerant
patients (n = 8) and non-tolerant patients (unsuccessful
wean (n = 6) and abnormal biopsies (n = 3))

| | Log₂ Fold | | |
|---|---|---|---|
| | Change | | |
| | (Tolerant vs | | |
| Gene Symbol | Non-Tolerant) | Padj | Gene Name |
| SELE | 1.67 | 0.00026 | E-selectin |
| XIST | −6.22 | 0.00088 | X-inactive specific transcript |
| EMB | −0.98 | 0.0013 | Embigin |
| TST | 0.37 | 0.0023 | Thiosulfate sulfurtransferase |
| UBD | −2.24 | 0.0026 | Ubiquitin D |
| LSP1 | −0.85 | 0.0053 | Lymphocyte-specific protein 1 |
| NR2F2 | −0.30 | 0.016 | Nuclear receptor subfamily 2 |
| | | | group F member 2 |
| AL162151.2 | −1.48 | 0.016 | Pseudogene similar to part of |
| | | | ribosomal protein L3 |
| TSPAN11 | 1.24 | 0.019 | Tetraspanin 11 |
| LGALS2 | −1.96 | 0.039 | Galectin 2 |
| IP6K3 | 2.41 | 0.041 | Inositol hexakisphosphate |
| | | | kinase 3 |
| CIITA | −0.67 | 0.041 | Class II major histocom- |
| | | | patibility complex |
| | | | transactivator |
| CXCL9 | −2.18 | 0.041 | C-X-C motif chemokine |
| | | | ligand 9 |
| GZMB | −1.42 | 0.046 | Granzyme B |
| S100A6 | −0.74 | 0.046 | S100 calcium binding protein |
| | | | A6 |
| AC136475.3 | 1.79 | 0.046 | Long non-coding RNA |

### Example 7

### Discussion

Solid organ transplantation is now recognized as a highly successful therapy for patients with end-stage disease (2). Yet despite this, the need for long-term IS leads to significant morbidity and mortality [2]. The ability to safely stop IS would presumably improve the long-term success of transplantation especially if it could be done early post-transplantation prior to the development of long-term complications such as renal dysfunction [2]. Although it is known that solid organ transplant patients and in particular liver transplant recipients may be operationally tolerant and can safely be weaned off IS, there is presently no reliable biomarker to identify these patients. The LITMUS study was a Phase 2a pilot study to examine whether a gene biomarker panel that was predictive of tolerance in preclinical heart and liver transplant models could identify operationally tolerant recipients. 28/69 (41%) liver transplant recipients were identified to have the putative PBMC tolerance biomarker and thus were candidates for IS withdrawal. Of these 28 patients, 23 had evaluable outcomes including eight patients who are operationally tolerant. Further analysis showed that patients who achieved tolerance had high baseline FOXP3/IFNγ allograft gene expression and high mRNA levels of E-selectin as detected by RNA-seq.

Compared to studies that do not rely on biomarkers to guide IS withdrawal, this PBMC biomarker approach appears to enrich for recipients who can successfully be weaned off IS. Here 8/14 (57%) of patients with the positive tolerance biomarker (FGL2/IFNγ≥1) and a normal liver biopsy were found to be operationally tolerant. This is in contrast to non-biomarker-guided studies where a combined 140/455 (30.8%) of adult liver transplant recipients were operationally tolerant [4-12]. Unlike LITMUS, many of these non-biomarker-guided studies used highly selected patients (non-autoimmune, non-viral replicative liver transplant recipients), which can improve success rates of IS withdrawal. Furthermore, patients had stopped IS at a time well after complications had developed. The utility of other PBMC biomarkers including FOXP3/IFNγ and TGFBI IFNγ was also examined. Although there was significant overlap in patients among these biomarkers (FIG. 5), the ratio of FGL2/IFNγ≥1 identified more patients who were operationally tolerant than either of the other two biomarkers. These results support the use of FGL2/IFNγ in future larger clinical studies.

The biomarker represents the ratio between an anti-inflammatory gene (FGL2) and the pro-inflammatory gene (IFNγ). FGL2 is known to be primarily secreted by Tregs and inhibits dendritic cell maturation following binding to its cognate receptor, FcγRIIb [27]. FGL2 has also been shown to inhibit a subset of effector CD8⁺ T cells that express FcγRIIb [28]. Recently, FGL2 has been shown to be an effector molecule of T follicular regulatory cells, which are known to limit antibody responses in germinal centres [29]. Without wishing to be bound by theory, upregulation of FGL2 gene expression may therefore serve to inhibit cellular and humoral allo- and autoimmune responses. IFNγ is a pro-inflammatory cytokine with important roles in T-cell activation and allograft rejection [30]. A high FGL2/IFNγ ratio therefore selects for low IFNγ gene expression and lower levels of T-cell activation. Transplant recipients who can be successfully weaned off IS presumably have less T-cell activation while they are immunosuppressed compared with non-tolerant patients and therefore have a higher FGL2/IFNγ gene ratio. The choice and level of calcineurin inhibitor and use of MMF was not different between tolerant and non-tolerant patients. However, there was an increased proportion of patients on MMF in the high FGL2/IFNγ biomarker group, which may reflect an inhibition of IFNγ by MMF as has been described previously [31].

In LITMUS, it is shown that monitoring of allograft gene expression may be critical to identifying operationally tolerant patients as the baseline liver allograft FOXP3/IFNγ gene ratio was higher in tolerant versus non-tolerant patients. This is consistent with previous studies showing that a higher intragraft Foxp3/IFNγ ratio correlated with tolerance versus rejection in preclinical transplant models [18, 19]. Importantly, the intragraft and not the PBMC FOXP3/IFNγ ratio was higher in tolerant versus non-tolerant patients, and FOXP3 gene expression by itself was not predictive of tolerance, consistent with prior studies showing no significant difference in graft infiltrating Tregs in baseline liver biopsies of tolerant and non-tolerant patients [17].

One of the strengths of the LITMUS study is that data from multiple time points post-IS withdrawal are provided. The data are supportive that operational tolerance is an active process involving peripheral regulation. An increase in both FOXP3 gene expression and numbers of Tregs by mass cytometry in the peripheral blood of operationally tolerant recipients were observed. This is similar to a previous report of increased numbers of CD4⁺CD25⁺ T cells and FOXP3 mRNA expression in the peripheral blood in liver transplant recipients who successfully discontinued immunosuppressive therapy [33]. Within the liver allograft, an increase in numbers of T cells in portal tracts and a proportionally larger increase in FOXP3⁺ Tregs were observed, similar to what has been observed in previous IS weaning trials [10, 26]. Although there was not an increase in intrahepatic FOXP3 gene expression during the development of operational tolerance, there was increase in gene expression for TGFB1, a known Treg effector molecule. These findings of the intrahepatic T-cell compartment point to active immune regulation involving immunoregulation by Tregs in the graft itself rather than a deletion of T-cell alloreactivity. However, the expansion of intragraft Treg may at least in part be related to withdrawal of calcineurin inhibitors, which are known to suppress Treg proliferation [34]. At this point, it cannot be distinguished between Treg expanding to control alloimmune responses versus expanding as a result of calcineurin withdrawal. In support of Treg playing an active role in transplantation tolerance are spontaneous models of liver transplant tolerance, which are characterized by an inflammatory infiltrate in the liver and an accompanying expansion of Tregs [19]. By post-operative day 100, there was near-complete resolution of the inflammatory infiltrate liver in this model (similar to liver biopsies of Patient Tol2), while operationally tolerant human liver grafts usually exhibit a long persistence of mild portal infiltrates [26]. That Tregs are necessary for tolerance in preclinical models has been confirmed as depletion of Tregs with an anti-CD25 antibody leads to rejection [35].

As part of the study design, liver biopsy samples were analysed with RNA-seq technology to identify additional genes associated with tolerance. Liver allografts from tolerant patients compared with non-tolerant patients expressed higher mRNA levels of SELE (gene for E-selectin) at baseline prior to IS withdrawal. E-selectin, which is an inducible adhesion molecule expressed by endothelial cells, plays an important role in recruitment of lymphocytes and neutrophils to sites of inflammation [36]. However, in the tolerant livers expression of E-selectin was increased in the presence of lower inflammatory gene expression. Interestingly, Tregs are reported to express ligands for E-selectin and thus expression of E-selectin may also be important for recruitment of Tregs [37, 38]. Furthermore, upregulation of E-selectin may be involved in the skewing the FOXP3/IFNγ ratio to tolerance through increased Treg recruitment. The gene for a tetraspanin protein (TSPAN11) was also upregulated in tolerant livers. Tetraspanins play an important role in cell adhesion and signalling, although little is known of the role of tetraspanin 11 in either the liver or immune function [39]. Additionally, inflammatory gene expression (UBD, LSP1, CIITA, CXCL9, and GZMB) was decreased in patients with successful IS withdrawal. Although IFNγ was not significantly decreased, a decrease in IFNγ inducible genes including UBD and CIITA in patients undergoing successful withdrawal was observed. Thus, the data lend support to using a ratio of gene expression (anti-inflammatory to pro-inflammatory) to identify liver transplant candidates for IS withdrawal. Based on results of the RNA-seq data, we plan to add SELE and TSPAN11 to the GeXP gene expression assay to determine if these additional genes will help in the identification of patients who can be weaned off IS.

In agreement with other studies, no patients with a history of autoimmune liver disease achieved operational tolerance [40]. Two patients with autoimmune liver disease did have the PBMC gene profile for tolerance but were found on liver biopsy to have histologic evidence of recurrent disease and were excluded from withdrawal of IS as per protocol. Therefore, the use of the biomarker even in these patients proved valuable in that histologic evidence of recurrent disease was detected despite normal liver biochemistry. Both of these patients were successfully treated with increased IS with resolution of liver inflammation.

Another important finding was that time after transplantation may be an important predictor of ability to wean IS, which is consistent with a larger study of operational tolerance in liver transplantation [10] and suggests host-graft adaptation over time [41]. Although age was not significantly higher in the group with successful wean, six of eight patients in this group were greater than 60 years old. In other studies, greater age is known to correlate with increased frequency of operational tolerance in liver transplantation, an observation which is likely related to diminished immune responses (immunosenescence) during the ageing process [42]. Of interest, in the present study, two patients who developed operational tolerance were young (less than 30 years of age). The PBMC biomarker may thus help in identifying young patients who are candidates for IS withdrawal. That young patients may develop tolerance is also supported by a previous study of IS withdrawal in paediatric liver transplant recipients [14].

Although the study was performed in liver transplant recipients, others have shown that spontaneous tolerance is achieved in recipients of other solid organs including heart, lung, kidney and pancreas transplanted patients.

In conclusion, the results of the study show that the PBMC biomarker (FGL2/IFNγ≥1) enriches the patient pool for liver transplant recipients who have developed operational tolerance and can be successfully weaned off IS. The data derived from LITMUS also shows that the combined use of the PBMC and a liver gene biomarker (FOXP3/IFNγ≥1) can increase the precision of the biomarker approach to identify tolerant patients. Furthermore, without being bound by theory, the immunological studies and gene expression studies over time point to active immune regulation involving Tregs as the mechanism underlying spontaneous operational tolerance.

### Example 8

The expression levels of the target genes are measured using MicroGEM's high performance RT-qPCR system. The MicroGEM SAL6830 cartridge and system is described for example in U.S. Pat. No. 11,465,145, the contents of which are incorporated for reference in their entirety. It can serve as an immune system dashboard (Immunometer). The system utilizes nanoparticle mediated cell capture with enzymatic lysis and extraction coupled with purification and integrated microfluidic PCR. Nanoparticles tethered to magnetic beads capture targeted PBMCs. In some cases, undifferentiated PBMCs are captured and then Tregs (CD4⁺ CD25⁺) and/or Transitional B cells (CD19⁺CD24hiCD38hi) are isolated using affinity ligands or antibody coated particles.

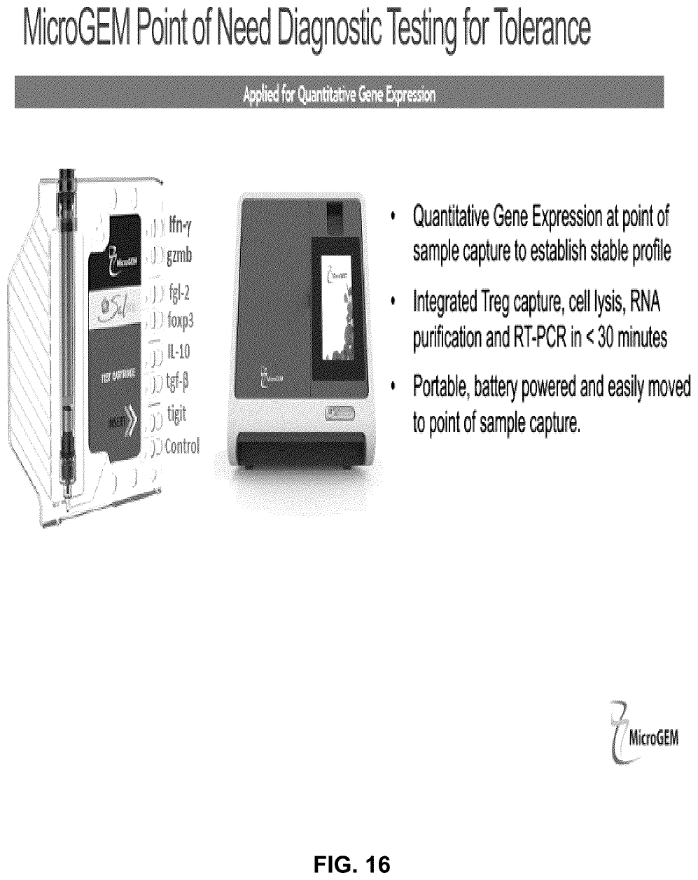

A cocktail of thermophilic enzymes operating at elevated temperature lyse cells, extract nucleic acids and render RNAses inactive. Thermally driven fluidics then drive extracts through purification chambers into 8 reaction chambers for ultrafast qPCR, resulting in analysis at point of capture of the specimen, where the stability of a gene expression profile is more easily maintained. A schematic of the MicroGEM Immunometer is shown in FIG. 16.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

1. Jain A, Reyes J, Kashyap R, et al. Long-term survival after liver transplantation in 4,000 consecutive patients at a single center. Ann Surg 2000, 232, 490-500.
2. Lechler R I, Sykes M, Thomson A W, Turka L A. Organ transplantation-how much of the promise has been realized? Nat Med 2005, 11, 605-13.
3. Thomson A W, Vionnet J, Sanchez-Fueyo A. Understanding, predicting and achieving liver transplant tolerance: from bench to bedside. Nat Rev Gastroenterol Hepatol 2020, 17, 719-39.
4. Mazariegos G V, Reyes J, Marino I R, et al. Weaning of immunosuppression in liver transplant recipients. Transplantation 1997, 63, 243-9.
5. Devlin J, Doherty D, Thomson L, et al. Defining the outcome of immunosuppression withdrawal after liver transplantation. Hepatology 1998, 27, 926-33.
6. Eason J D, Cohen A J, Nair S, Alcantera T, Loss G E. Tolerance: is it worth the risk? Transplantation 2005, 79, 1157-9.
7. Tryphonopoulos P, Tzakis A G, Weppler D, et al. The role of donor bone marrow infusions in withdrawal of immunosuppression in adult liver allotransplantation. Am J Transplant 2005, 5, 608-13.
8. Tisone G, Orlando G, Cardillo A, et al. Complete weaning off immunosuppression in HCV liver transplant recipients is feasible and favourably impacts on the progression of disease recurrence. J Hepatol 2006, 44, 702-9.
9. Pons J A, Ramirez P, Revilla-Nuin B, et al. Immunosuppression withdrawal improves long-term metabolic parameters, cardiovascular risk factors and renal function in liver transplant patients. Clin Transplant 2009, 23, 329-36.
10. Benitez C, Londo\ddot{n}o M C, Miquel R, et al. Prospective multicenter clinical trial of immunosuppressive drug withdrawal in stable adult liver transplant recipients. Hepatology 2013, 58, 1824-35.
11. de la Garza R G, Sarobe P, Merino J, et al. Trial of complete weaning from immunosuppression for liver transplant recipients: factors predictive of tolerance. Liver Transplant 2013, 19, 937-44.
12. Takatsuki M, Uemoto S, Inomata Y, et al. Weaning of immunosuppression in living donor liver transplant recipients. Transplantation 2001, 72, 449-54.
13. Appenzeller-Herzog C, Hartleif S, Vionnet J. Clinical parameters and biomarkers predicting spontaneous operational tolerance after liver transplantation: a scoping review. Am J Transplant 2021, 21, 3312-23.
14. Feng S, Ekong U D, Lobritto S J, et al. Complete immunosuppression withdrawal and subsequent allograft function among pediatric recipients of parental living donor liver transplants. JAMA 2012, 307, 283-93.
15. Martinez-Llordella M, Puig-Pey I, Orlando G, et al. Multiparameter immune profiling of operational tolerance in liver transplantation. Am J Transplant 2007, 7, 309-19.
16. Li L, Wozniak L J, Rodder S, et al. A common peripheral blood gene set for diagnosis of operational tolerance in pediatric and adult liver transplantation. Am J Transplant 2012, 12, 1218-28.
17. Bohne F, Martinez-Llordella M, Lozano J J, et al. Intra-graft expression of genes involved in iron homeostasis predicts the development of operational tolerance in human liver transplantation. J Clin Invest 2012, 122, 368-82.
18. Urbanellis P, Shyu W, Khattar R, et al. The regulatory T cell effector molecule fibrinogen-like protein 2 is necessary for the development of rapamycin-induced tolerance to fully MHC-mismatched murine cardiac allografts. Immunology 2015, 144, 91-106.
19. Xie L, Ichimaru N, Morita M, et al. Identification of a novel biomarker gene set with sensitivity and specificity for distinguishing between allograft rejection and tolerance. Liver Transpl 2012, 18, 444-54.
20. Wood K J. Regulatory T cells in transplantation. Transplant Proc 2011, 43, 2135-6.
21. Demetris A J, Bellamy C, Hübscher S G, et al. 2016 Comprehensive update of the Banff working group on liver allograft pathology: introduction of antibody-mediated rejection. Am J Transplant 2016, 16, 2816-35.
22. Banff Working Group on Liver Allograft P. Importance of liver biopsy findings in immunosuppression management: biopsy monitoring and working criteria for patients with operational tolerance. Liver Transplant 2012, 18, 1154-70.
23. Livak K J, Schmittgen T D. Analysis of relative gene expression data using real-time quantitative PCR and the 2(−delta delta C(T)) method. Methods 2001, 25, 402-8.
24. Streitz M, Miloud T, Kapinsky M, et al. Standardization of whole blood immune phenotype monitoring for clinical trials: panels and methods from the ONE study. Transplant Res 2013, 2, 17.
25. Taubert R, Hardtke-Wolenski M, Noyan F, et al. Intrahepatic regulatory T cells in autoimmune hepatitis are associated with treatment response and depleted with current therapies. J Hepatol 2014, 61, 1106-14.
26. Taubert R, Danger R, Londoño M C, et al. Hepatic infiltrates in operational tolerant patients after liver transplantation show enrichment of regulatory T cells before proinflammatory genes are downregulated. Am J Transplant 2016, 16, 1285-93.
27. Chruscinski A, Sadozai H, Rojas-Luengas V, et al. Role of regulatory T cells (Treg) and the Treg effector molecule fibrinogen-like protein 2 in alloimmunity and autoimmunity. Rambam Maimonides Med J 2015, 6, e0024.
28. Morris A B, Farley C R, Pinelli D F, et al. Signaling through the inhibitory Fc receptor FcγRIIB induces CD8+ T cell apoptosis to limit T cell immunity. Immunity 2020, 52, 136-50.e6.
29. Sungnak W, Wagner A, Kowalczyk M S, et al. T follicular regulatory cell-derived fibrinogen-like protein 2 regulates production of autoantibodies and induction of systemic autoimmunity. J Immunol 2020, 205, 3247-62.
30. Hidalgo L G, Halloran P F. Role of IFN-gamma in allograft rejection. Crit Rev Immunol 2002, 22, 317-49.
31. Lui S L, Ramassar V, Urmson J, Halloran P F. Mycophenolate mofetil reduces production of interferon-dependent major histocompatibility complex induction during allograft rejection, probably by limiting clonal expansion. Transpl Immunol 1998, 6, 23-32.
32. Cherukuri A, Salama A D, Mehta R, et al. Transitional B cell cytokines predict renal allograft outcomes. Sci Transl Med 2021, 13, eabe4929.
33. Pons J A, Revilla-Nuin B, Baroja-Mazo A, et al. FoxP3 in peripheral blood is associated with operational tolerance in liver transplant patients during immunosuppression withdrawal. Transplantation 2008, 86, 1370-8.
34. Furukawa A, Wisel S A, Tang Q. Impact of immune-modulatory drugs on regulatory T cell. Transplantation 2016, 100, 2288-300.
35. Li W, Carper K, Liang Y, et al. Anti-CD25 mAb administration prevents spontaneous liver transplant tolerance. Transplant Proc 2006, 38, 3207-8.
36. McEver R P. Selectins: initiators of leucocyte adhesion and signaling at the vascular wall. Cardiovasc Res 2015, 107, 331-9.
37. Abeynaike L D, Deane J A, Westhorpe C L, et al. Regulatory T cells dynamically regulate selectin ligand function during multiple challenge contact hypersensitivity. J Immunol 2014, 193, 4934-44.
38. Zhang N, Schröppel B, Lal G, et al. Regulatory T cells sequentially migrate from inflamed tissues to draining lymph nodes to suppress the alloimmune response. Immunity 2009, 30, 458-69.
39. Charrin S, Jouannet S, Boucheix C, Rubinstein E. Tetraspanins at a glance. J Cell Sci 2014, 127, 3641-8.
40. Levitsky J. Operational tolerance: past lessons and future prospects. Liver Transpl 2011, 17, 222-32.
41. Dai H, Zheng Y, Thomson A W, Rogers N M. Transplant tolerance induction: insights from the liver. Front Immunol 2020, 11, 1044.
42. Goronzy J J, Weyand C M. Mechanisms underlying T cell ageing. Nat Rev Immunol 2019, 19, 573-83.

1. A method of predicting operational tolerance in a transplant patient who is on an immunosuppressant, the method comprising:
determining a peripheral blood mononuclear cell (PBMC) ratio of the expression levels of an anti-inflammatory gene to a pro-inflammatory gene in PBMCs from the patient;
wherein the anti-inflammatory gene is selected from the group consisting of FGL2, FOXP3, IL10, TIGIT, LAG3 and TGFB;
wherein the pro-inflammatory gene is selected from the group consisting of IFNγ and GZMB; and
wherein a PBMC ratio of ≥1 is indicative that the patient will achieve operational tolerance.

2. The method of claim 1, wherein the transplant is a solid organ transplant, optionally a heart, kidney, pancreas, lung or liver transplant.

3. The method of claim 2, wherein the transplant is a liver transplant and the transplant patient is previously diagnosed with hepatitis C virus (HCV) cirrhosis, alcoholic cirrhosis, autoimmune disease, genetic liver disease, fulminant hepatic failure (FHF), and/or non-alcoholic steatohepatitis (NASH).

4. The method of claim 1, wherein the PBMCs are Tregs or transitional B cells.

5. The method of claim 1, wherein determining the PBMC ratio comprises measuring the expression levels of the anti-inflammatory gene and the pro-inflammatory gene in PBMCs, optionally wherein measuring the expression levels comprises performing quantitative PCR, optionally ultra fast qPCR.

6. The method of claim 1, wherein the anti-inflammatory gene is FGL2 and/or wherein the pro-inflammatory gene is IFNγ.

7. The method of claim 1, further comprising:
determining an intragraft ratio of an anti-inflammatory gene to a pro-inflammatory gene in a graft sample of the patient;
wherein a PBMC ratio of ≥1 combined with an intragraft ratio of ≥1 is indicative that the patient will achieve operational tolerance.

8. The method of claim 7, wherein the graft sample is a liver biopsy sample.

9. The method of claim 7, wherein the anti-inflammatory gene for the intragraft ratio is FOXP3 and/or wherein the pro-inflammatory gene for the intragraft ratio is INFγ.

10. The method of claim 1, further comprising reducing the dosage of the immunosuppressant in the patient.

11. A method of identifying a transplant patient on an immunosuppressant as a candidate for reducing the dosage of the immunosuppressant, the method comprising:
determining a peripheral blood mononuclear cell (PBMC) ratio of the expression levels of an anti-inflammatory gene to a pro-inflammatory gene in PBMCs from the patient;
wherein the anti-inflammatory gene is selected from the group consisting of FGL2, FOXP3, IL10, TIGIT, LAG3 and TGFB;
wherein the pro-inflammatory gene is selected from the group consisting of IFNγ and GZMB;
wherein if the PBMC ratio is ≥1, then the patient is a candidate for reducing the dosage of the immunosuppressant.

12. The method of claim 11, wherein the transplant is a solid organ transplant, optionally a heart, kidney, pancreas, lung or liver transplant.

13. The method of claim 12, wherein the transplant is a liver transplant and the transplant patient is previously diagnosed with hepatitis C virus (HCV) cirrhosis, alcoholic cirrhosis, autoimmune disease, genetic liver disease, fulminant hepatic failure (FHF), and/or non-alcoholic steatohepatitis (NASH).

13. The method of claim 11, wherein the PBMCs are Tregs or transitional B cells.

14. The method of claim 11, wherein determining the PBMC ratio comprises measuring the expression levels of the anti-inflammatory gene and the pro-inflammatory gene, optionally wherein measuring the expression levels comprises performing quantitative PCR, optionally ultrafast quantitative PCR.

15. The method of claim 11, wherein the anti-inflammatory gene is FGL2 and/or the pro-inflammatory gene is IFNγ.

16. The method of claim 11, further comprising:
determining an intragraft ratio of an anti-inflammatory gene to a pro-inflammatory gene in a graft sample, optionally a liver biopsy sample, of the patient;
wherein if the PBMC ratio is ≥1 and the intragraft ratio is ≥1, then the patient is a candidate for reducing the dosage of the immunosuppressant.

17. The method of claim 16, wherein the anti-inflammatory gene for the intragraft ratio is FOXP3 and/or wherein the pro-inflammatory gene for the intragraft ratio is IFNγ.

18. The method of claim 11, wherein the PBMC ratio is a ratio of the expression levels of FGL2 to IFNγ in PBMCs, and wherein the intragraft ratio is a ratio of the expression levels of FOXP3 to IFNγ in the graft sample.

19. The method of claim 11, further comprising reducing the dosage of immunosuppressant in the patient, optionally wherein reducing the dosage of immunosuppressant is complete cessation of immunosuppressant.

20. A kit comprising:
reagents for measuring the expression level of at least one anti-inflammatory gene, wherein the anti-inflammatory gene is selected from the group consisting of FGL2, FOXP3, IL10, TIGIT, LAG3 and TGFB; and
reagents for measuring the expression level of at least one pro-inflammatory gene, wherein the pro-inflammatory gene is selected from the group consisting of IFNγ and GZMB.
